SEQUENCE LISTING (9 sequences)

SEQ ID NO: 1 - DNA, 4314 bp, Homo sapiens
atggaggatatcgacataggaaaagagtatatcatccccagtcctgggtatagaagtgtgagggagagaaccagcacttctgggacgcacagagaccgtgaagattccaagttcaggagaactcgaccgttggaatgccaagatgccttggaaacagcagcccgagccgagggcctctctcttgatgcctccatgcattctcagctcagaatcctggatgaggagcatcccaagggaaagtaccatcatggcttgagtgctctgaagcccatccggactacttccaaacaccagcacccagtggacaatgctgggcttttttcctgtatgactttttcgtggctttcttctctggcccgtgtggcccacaagaagggggagctctcaatggaagacgtgtggtctctgtccaagcacgagtcttctgacgtgaactgcagaagactagagagactgtggcaagaagagctgaatgaagttgggccagacgctgcttccctgcgaagggttgtgtggatcttctgccgcaccaggctcatcctgtccatcgtgtgcctgatgatcacgcagctggctggcttcagtggaccagccttcatggtgaaacacctcttggagtatacccaggcaacagagtctaacctgcagtacagcttgttgttagtgctgggcctcctcctgacggaaatcgtgcggtcttggtcgcttgcactgacttgggcattgaattaccgaaccggtgtccgcttgcggggggccatcctaaccatggcatttaagaagatccttaagttaaagaacattaaagagaaatccctgggtgagctcatcaacatttgctccaacgatgggcagagaatgtttgaggcagcagccgttggcagcctgctggctggaggacccgttgttgccatcttaggcatgatttataatgtaattattctgggaccaacaggcttcctgggatcagctgtttttatcctcttttacccagcaatgatgtttgcatcacggctcacagcatatttcaggagaaaatgcgtggccgccacggatgaacgtgtccagaagatgaatgaagttcttacttacattaaatttatcaaaatgtatgcctgggtcaaagcattttctcagagtgttcagaaaatccgcgaggaggagcgtcggatattggaaaaagccgggtacttccagagcatcactgtgggtgtggctcccattgtggtggtgattgccagcgtggtgaccttctctgttcatatgaccctgggcttcgatctgacagcagcacaggctttcacagtggtgacagtcttcaattccatgacttttgctttgaaagtaacaccgttttcagtaaagtccctctcagaagcctcagtggctgttgacagatttaagagtttgtttctaatggaagaggttcacatgataaagaacaaaccagccagtcctcacatcaagatagagatgaaaaatgccaccttggcatgggactcctcccactccagtatccagaactcgcccaagctgacccccaaaatgaaaaaagacaagagggcttccaggggcaagaaagagaaggtgaggcagctgcagcgcactgagcatcaggcggtgctggcagagcagaaaggccacctcctcctggacagtgacgagcggcccagtcccgaagaggaagaaggcaagcacatccacctgggccacctgcgcttacagaggacactgcacagcatcgatctggagatccaagagggtaaactggttggaatctgtggcagtgtgggaagtggaaaaacctctctcatttcagccattttaggccagatgacgcttctagagggcagcattgcaatcagtggaaccttcgcttatgtggcccagcaggcctggatcctcaatgctactctgagagacaacatcctgtttgggaaggaatatgatgaagaaagatacaactctgtgctgaacagctgctgcctgaggcctgacctggccattcttcccagcagcgacctgacggagattggagagcgaggagccaacctgagcggtgggcagcgccagaggatcagccttgcccgggccttgtatagtgacaggagcatctacatcctggacgaccccctcagtgccttagatgcccatgtgggcaaccacatcttcaatagtgctatccggaaacatctcaagtccaagacagttctgtttgttacccaccagttacagtacctggttgactgtgatgaagtgatcttcatgaaagagggctgtattacggaaagaggcacccatgaggaactgatgaatttaaatggtgactatgctaccatttttaataacctgttgctgggagagacaccgccagttgagatcaattcaaaaaaggaaaccagtggttcacagaagaagtcacaagacaagggtcctaaaacaggatcagtaaagaaggaaaaagcagtaaagccagaggaagggcagcttgtgcagctggaagagaaagggcagggttcagtgccctggtcagtatatggtgtctacatccaggctgctgggggccccttggcattcctggttattatggcccttttcatgctgaatgtaggcagcaccgccttcagcacctggtggttgagttactggatcaagcaaggaagcgggaacaccactgtgactcgagggaacgagacctcggtgagtgacagcatgaaggacaatcctcatatgcagtactatgccagcatctacgccctctccatggcagtcatgctgatcctgaaagccattcgaggagttgtctttgtcaagggcacgctgcgagcttcctcccggctgcatgacgagcttttccgaaggatccttcgaagccctatgaagttttttgacacgacccccacagggaggattctcaacaggttttccaaagacatggatgaagttgacgtgcggctgccgttccaggccgagatgttcatccagaacgttatcctggtgttcttctgtgtgggaatgatcgcaggagtcttcccgtggttccttgtggcagtggggccccttgtcatcctcttttcagtcctgcacattgtctccagggtcctgattcgggagctgaagcgtctggacaatatcacgcagtcacctttcctctcccacatcacgtccagcatacagggccttgccaccatccacgcctacaataaagggcaggagtttctgcacagataccaggagctgctggatgacaaccaagctcctttttttttgtttacgtgtgcgatgcggtggctggctgtgcggctggacctcatcagcatcgccctcatcaccaccacggggctgatgatcgttcttatgcacgggcagattcccccagcctatgcgggtctcgccatctcttatgctgtccagttaacggggctgttccagtttacggtcagactggcatctgagacagaagctcgattcacctcggtggagaggatcaatcactacattaagactctgtccttggaagcacctgccagaattaagaacaaggctccctcccctgactggccccaggagggagaggtgacctttgagaacgcagagatgaggtaccgagaaaacctccctctcgtcctaaagaaagtatccttcacgatcaaacctaaagagaagattggcattgtggggcggacaggatcagggaagtcctcgctggggatggccctcttccgtctggtggagttatctggaggctgcatcaagattgatggagtgagaatcagtgatattggccttgccgacctccgaagcaaactctctatcattcctcaagagccggtgctgttcagtggcactgtcagatcaaatttggaccccttcaaccagtacactgaagaccagatttgggatgccctggagaggacacacatgaaagaatgtattgctcagctacctctgaaacttgaatctgaagtgatggagaatggggataacttctcagtgggggaacggcagctcttgtgcatagctagagccctgctccgccactgtaagattctgattttagatgaagccacagctgccatggacacagagacagacttattgattcaagagaccatccgagaagcatttgcagactgtaccatgctgaccattgcccatcgcctgcacacggttctaggctccgataggattatggtgctggcccagggacaggtggtggagtttgacaccccatcggtccttctgtccaacgacagttcccgattctatgccatgtttgctgctgcagagaacaaggtcgctgtcaagggctga

SEQ ID NO: 2 - protein, 1437 aa, Homo sapiens
MEDIDIGKEYIIPSPGYRSVRERTSTSGTHRDREDSKFRRTRPLECQDALETAARAEGLSLDASMHSQLRILDEEHPKGKYHHGLSALKPIRTTSKHQHPVDNAGLFSCMTFSWLSSLARVAHKKGELSMEDVWSLSKHESSDVNCRRLERLWQEELNEVGPDAASLRRVVWIFCRTRLILSIVCLMITQLAGFSGPAFMVKHLLEYTQATESNLQYSLLLVLGLLLTEIVRSWSLALTWALNYRTGVRLRGAILTMAFKKILKLKNIKEKSLGELINICSNDGQRMFEAAAVGSLLAGGPVVAILGMIYNVIILGPTGFLGSAVFILFYPAMMFASRLTAYFRRKCVAATDERVQKMNEVLTYIKFIKMYAWVKAFSQSVQKIREEERRILEKAGYFQSITVGVAPIVVVIASVVTFSVHMTLGFDLTAAQAFTVVTVFNSMTFALKVTPFSVKSLSEASVAVDRFKSLFLMEEVHMIKNKPASPHIKIEMKNATLAWDSSHSSIQNSPKLTPKMKKDKRASRGKKEKVRQLQRTEHQAVLAEQKGHLLLDSDERPSPEEEEGKHIHLGHLRLQRTLHSIDLEIQEGKLVGICGSVGSGKTSLISAILGQMTLLEGSIAISGTFAYVAQQAWILNATLRDNILFGKEYDEERYNSVLNSCCLRPDLAILPSSDLTEIGERGANLSGGQRQRISLARALYSDRSIYILDDPLSALDAHVGNHIFNSAIRKHLKSKTVLFVTHQLQYLVDCDEVIFMKEGCITERGTHEELMNLNGDYATIFNNLLLGETPPVEINSKKETSGSQKKSQDKGPKTGSVKKEKAVKPEEGQLVQLEEKGQGSVPWSVYGVYIQAAGGPLAFLVIMALFMLNVGSTAFSTWWLSYWIKQGSGNTTVTRGNETSVSDSMKDNPHMQYYASIYALSMAVMLILKAIRGVVFVKGTLRASSRLHDELFRRILRSPMKFFDTTPTGRILNRFSKDMDEVDVRLPFQAEMFIQNVILVFFCVGMIAGVFPWFLVAVGPLVILFSVLHIVSRVLIRELKRLDNITQSPFLSHITSSIQGLATIHAYNKGQEFLHRYQELLDDNQAPFFLFTCAMRWLAVRLDLISIALITTTGLMIVLMHGQIPPAYAGLAISYAVQLTGLFQFTVRLASETEARFTSVERINHYIKTLSLEAPARIKNKAPSPDWPQEGEVTFENAEMRYRENLPLVLKKVSFTIKPKEKIGIVGRTGSGKSSLGMALFRLVELSGGCIKIDGVRISDIGLADLRSKLSIIPQEPVLFSGTVRSNLDPFNQYTEDQIWDALERTHMKECIAQLPLKLESEVMENGDNFSVGERQLLCIARALLRHCKILILDEATAAMDTETDLLIQETIREAFADCTMLTIAHRLHTVLGSDRIMVLAQGQVVEFDTPSVLLSNDSSRFYAMFAAAENKVAVKG

SEQ ID NO: 3 - DNA, 2687 bp, Homo sapiens
taccgagctcggatccactagtccagtgtggtggaattgcccttgccaccatggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctacccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaagtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaagctgagtgagaagcaggaggtgaagaaggagctggtgacagagatcctgacggcactgggcctgatgtcgtgctcccgcacgaggacagccctgctctctggcgggcagaggaagcgtctggccatcgccctggagctggtcaacaacccgcctgtcatgttctttgatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggcgtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgatcctgacgatctatggcatggagcgaggagacctgacatgtttagaggaacgctgcccgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatagaggcttgccccagcctgtaccccagcccctgcagcaggaagcccccagtcccagccctttgggactgttttaaccttatagacttgggcactggttcctggcggggctatcctctcctcccttggctcctccacaggctggctgtcggactgcgctcccagcctgggctctgggagtgggggctccagccctccccactatgcccaggagtcttcccaagttgatgcggtttgtagcttcctccctactctctccaacacctgcatgcaaagactactgggaggctgctgcctccttcctgcccatggcaccctcctctgctgtctgcctgggagccctaggctctctagggccccacttacaactgaccaaagtggccccctctkggggtccccaccacacaagtgtttgtaaactgggctgctataaggttggagttccagggctgggccctggtggagtccactggaagtcccatcatggatgttgaaatggacagggaaggactctggaagtctcttcctcctcctcctcttctctccacccctagaccctggctgacttggacaatctgccaggacagaagctggggttttctgtctaggtcaccactcccaatcctgggggrttggagrggcctggggstgtgggrtgscccatccccctccccatcacctttggtgggggsagggcctg

SEQ ID NO: 4 - protein, 646 aa, Homo sapiens
MAEKALEAVGCGLGPGAVAMAVTLEDGAEPPVLTTHLKKVENHITEAQRFSHLPKRSAVDIEFVELSYSVREGPCWRKRGYKTLLKCLSGKFCRRELIGIMGPSGAGKSTFMNILAGYRESGMKGQILVNGRPRELRTFRKMSCYIMQDDMLLPHLTVLEAMMVSANLKLSEKQEVKKELVTEILTALGLMSCSRTRTALLSGGQRKRLAIALELVNNPPVMFFDEPTSGLDSASCFQVVSLMKSLAQGGRTIICTIHQPSAKLFEMFDKLYILSQGQCIFKGVVTNLIPYLKGLGLHCPTYHNPADFIIEVASGEYGDLNPMLFRAVQNGLCAMAEKKSSPEKNEVPAPCPPCPPEVDPIESHTFATSTLTQFCILFKRTFLSILRDTVLTHLRFMSHVVIGVLIGLLYLHIGDDASKVFNNTGCLFFSMLFLMFAALMPTVLTFPLEMAVFMREHLNYWYSLKAYYLAKTMADVPFQVVCPVVYCSIVYWMTGQPAETSRFLLFSALATATALVAQSLGLLIGAASNSLQVATFVGPVTAIPVLLFSGFFVSFKTIPTYLQWSSYLSYVRYGFEGVILTIYGMERGDLTCLEERCPFREPQSILRALDVEDAKLYMDFLVLGIFFLALRLLAYLVLRYRVKSER

SEQ ID NO: 5 - DNA, 3978 bp, Homo sapiens
atggaggatatcgacataggaaaagagtatatcatccccagtcctgggtatagaagtgtgagggagagaaccagcacttctgggacgcacagagaccgtgaagattccaagttcaggagaactcgaccgttggaatgccaagatgccttggaaacagcagcccgagccgagggcctctctcttgatgcctccatgcattctcagctcagaatcctggatgaggagcatcccaagggaaagtaccatcatggcttgagtgctctgaagcccatccggactacttccaaacaccagcacccagtggacaatgctgggcttttttcctgtatgactttttcgtggctttcttctctggcccgtgtggcccacaagaagggggagctctcaatggaagacgtgtggtctctgtccaagcacgagtcttctgacgtgaactgcagaagactagagagactgtggcaagaagagctgaatgaagttgggccagacgctgcttccctgcgaagggttgtgtggatcttctgccgcaccaggctcatcctgtccatcgtgtgcctgatgatcacgcagctggctggcttcagtggaccagccttcatggtgaaacacctcttggagtatacccaggcaacagagtctaacctgcagtacagcttgttgttagtgctgggcctcctcctgacggaaatcgtgcggtcttggtcgcttgcactgacttgggcattgaattaccgaaccggtgtccgcttgcggggggccatcctaaccatggcatttaagaagatccttaagttaaagaacattaaagagaaatccctgggtgagctcatcaacatttgctccaacgatgggcagagaatgtttgaggcagcagccgttggcagcctgctggctggaggacccgttgttgccatcttaggcatgatttataatgtaattattctgggaccaacaggcttcctgggatcagctgtttttatcctcttttacccagcaatgatgtttgcatcacggctcacagcatatttcaggagaaaatgcgtggccgccacggatgaacgtgtccagaagatgaatgaagttcttacttacattaaatttatcaaaatgtatgcctgggtcaaagcattttctcagagtgttcagaaaatccgcgaggaggagcgtcggatattggaaaaagccgggtacttccagagcatcactgtgggtgtggctcccattgtggtggtgattgccagcgtggtgaccttctctgttcatatgaccctgggcttcgatctgacagcagcacaggctttcacagtggtgacagtcttcaattccatgacttttgctttgaaagtaacaccgttttcagtaaagtccctctcagaagcctcagtggctgttgacagatttaagagtttgtttctaatggaagaggttcacatgataaagaacaaaccagccagtcctcacatcaagatagagatgaaaaatgccaccttggcatgggactcctcccactccagtatccagaactcgcccaagctgacccccaaaatgaaaaaagacaagagggcttccaggggcaagaaagagaaggtgaggcagctgcagcgcactgagcatcaggcggtgctggcagagcagaaaggccacctcctcctggacagtgacgagcggcccagtcccgaagaggaagaaggcaagcacatccacctgggccacctgcgcttacagaggacactgcacagcatcgatctggagatccaagagggtaaactggttggaatctgtggcagtgtgggaagtggaaaaacctctctcatttcagccattttaggccagatgacgcttctagagggcagcattgcaatcagtggaaccttcgcttatgtggcccagcaggcctggatcctcaatgctactctgagagacaacatcctgtttgggaaggaatatgatgaagaaagatacaactctgtgctgaacagctgctgcctgaggcctgacctggccattcttcccagcagcgacctgacggagattggagagcgaggagccaacctgagcggtgggcagcgccagaggatcagccttgcccgggccttgtatagtgacaggagcatctacatcctggacgaccccctcagtgccttagatgcccatgtgggcaaccacatcttcaatagtgctatccggaaacatctcaagtccaagacagttctgtttgttacccaccagttacagtacctggttgactgtgatgaagtgatcttcatgaaagagggctgtattacggaaagaggcacccatgaggaactgatgaatttaaatggtgactatgctaccatttttaataacctgttgctgggagagacaccgccagttgagatcaattcaaaaaaggaaaccagtggttcacagaagaagtcacaagacaagggtcctaaaacaggatcagtaaagaaggaaaaagcagtaaagccagaggaagggcagcttgtgcagctggaagagaaagggcagggttcagtgccctggtcagtatatggtgtctacatccaggctgctgggggccccttggcattcctggttattatggcccttttcatgctgaatgtaggcagcaccgccttcagcacctggtggttgagttactggatcaagcaaggaagcgggaacaccactgtgactcgagggaacgagacctcggtgagtgacagcatgaaggacaatcctcatatgcagtactatgccagcatctacgccctctccatggcagtcatgctgatcctgaaagccattcgaggagttgtctttgtcaagggcacgctgcgagcttcctcccggctgcatgacgagcttttccgaaggatccttcgaagccctatgaagttttttgacacgacccccacagggaggattctcaacaggttttccaaagacatggatgaagttgacgtgcggctgccgttccaggccgagatgttcatccagaacgttatcctggtgttcttctgtgtgggaatgatcgcaggagtcttcccgtggttccttgtggcagtggggccccttgtcatcctcttttcagtcctgcacattgtctccagggtcctgattcgggagctgaagcgtctggacaatatcacgcagtcacctttcctctcccacatcacgtccagcatacagggccttgccaccatccacgcctacaataaagggcaggagtttctgcacagataccaggagctgctggatgacaaccaagctcctttttttttgtttacgtgtgcgatgcggtggctggctgtgcggctggacctcatcagcatcgccctcatcaccaccacggggctgatgatcgttcttatgcacgggcagattcccccagcctatgcgggtctcgccatctcttatgctgtccagttaacggggctgttccagtttacggtcagactggcatctgagacagaagctcgattcacctcggtggagaggatcaatcactacattaagactctgtccttggaagcacctgccagaattaagaacaaggctccctcccctgactggccccaggagggagaggtgacctttgagaacgcagagatgaggtaccgagaaaacctccctctcgtcctaaagaaagtatccttcacgatcaaacctaaagagaagattggcattgtggggcggacaggatcagggaagtcctcgctggggatggccctcttccgtctggtggagttatctggaggctgcatcaagattgatggagtgagaatcagtgatattggccttgccgacctccgaagcaaactctctatcattcctcaagagccggtgctgttcagtggcactgtcagatcaaatttggaccccttcaaccagtacactgaagaccagatttgggatgccctggagaggacacacatgaaagaatgtatcgaggccggcaaggccgaatccagacatgataagatacattga

SEQ ID NO: 6 - protein, 1325 aa, Homo sapiens
MEDIDIGKEYIIPSPGYRSVRERTSTSGTHRDREDSKFRRTRPLECQDALETAARAEGLSLDASMHSQLRILDEEHPKGKYHHGLSALKPIRTTSKHQHPVDNAGLFSCMTFSWLSSLARVAHKKGELSMEDVWSLSKHESSDVNCRRLERLWQEELNEVGPDAASLRRVVWIFCRTRLILSIVCLMITQLAGFSGPAFMVKHLLEYTQATESNLQYSLLLVLGLLLTEIVRSWSLALTWALNYRTGVRLRGAILTMAFKKILKLKNIKEKSLGELINICSNDGQRMFEAAAVGSLLAGGPVVAILGMIYNVIILGPTGFLGSAVFILFYPAMMFASRLTAYFRRKCVAATDERVQKMNEVLTYIKFIKMYAWVKAFSQSVQKIREEERRILEKAGYFQSITVGVAPIVVVIASVVTFSVHMTLGFDLTAAQAFTVVTVFNSMTFALKVTPFSVKSLSEASVAVDRFKSLFLMEEVHMIKNKPASPHIKIEMKNATLAWDSSHSSIQNSPKLTPKMKKDKRASRGKKEKVRQLQRTEHQAVLAEQKGHLLLDSDERPSPEEEEGKHIHLGHLRLQRTLHSIDLEIQEGKLVGICGSVGSGKTSLISAILGQMTLLEGSIAISGTFAYVAQQAWILNATLRDNILFGKEYDEERYNSVLNSCCLRPDLAILPSSDLTEIGERGANLSGGQRQRISLARALYSDRSIYILDDPLSALDAHVGNHIFNSAIRKHLKSKTVLFVTHQLQYLVDCDEVIFMKEGCITERGTHEELMNLNGDYATIFNNLLLGETPPVEINSKKETSGSQKKSQDKGPKTGSVKKEKAVKPEEGQLVQLEEKGQGSVPWSVYGVYIQAAGGPLAFLVIMALFMLNVGSTAFSTWWLSYWIKQGSGNTTVTRGNETSVSDSMKDNPHMQYYASIYALSMAVMLILKAIRGVVFVKGTLRASSRLHDELFRRILRSPMKFFDTTPTGRILNRFSKDMDEVDVRLPFQAEMFIQNVILVFFCVGMIAGVFPWFLVAVGPLVILFSVLHIVSRVLIRELKRLDNITQSPFLSHITSSIQGLATIHAYNKGQEFLHRYQELLDDNQAPFFLFTCAMRWLAVRLDLISIALITTTGLMIVLMHGQIPPAYAGLAISYAVQLTGLFQFTVRLASETEARFTSVERINHYIKTLSLEAPARIKNKAPSPDWPQEGEVTFENAEMRYRENLPLVLKKVSFTIKPKEKIGIVGRTGSGKSSLGMALFRLVELSGGCIKIDGVRISDIGLADLRSKLSIIPQEPVLFSGTVRSNLDPFNQYTEDQIWDALERTHMKECIEAGKAESRHDKIH

SEQ ID NO: 7 - DNA, 2687 bp, Homo sapiens
taccgagctcggatccactagtccagtgtggtggaattgcccttgccaccatggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctacccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaggtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaagctgagtgagaagcaggaggtgaagaaggagctggtgacagagatcctgacggcactgggcctgatgtcgtgctcccgcacgaggacagccctgctctctggcgggcagaggaagcgtctggccatcgccctggagctggtcaacaacccgcctgtcatgttctttaatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggcgtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgatcctgacgatctatggcatggagcgaggagacctgacatgtttagaggaacgctgcccgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatagaggcttgccccagcctgtaccccagcccctgcagcaggaagcccccagtcccagccctttgggactgttttaaccttatagacttgggcactggttcctggcggggctatcctctcctcccttggctcctccacaggctggctgtcggactgcgctcccagcctgggctctgggagtgggggctccagccctccccactatgcccaggagtcttcccaagttgatgcggtttgtagcttcctccctactctctccaacacctgcatgcaaagactactgggaggctgctgcctccttcctgcccatggcaccctcctctgctgtctgcctgggagccctaggctctctagggccccacttacaactgaccaaagtggccccctctkggggtccccaccacacaagtgtttgtaaactgggctgctataaggttggagttccagggctgggccctggtggagtccactggaagtcccatcatggatgttgaaatggacagggaaggactctggaagtctcttcctcctcctcctcttctctccacccctagaccctggctgacttggacaatctgccaggacagaagctggggttttctgtctaggtcaccactcccaatcctgggggrttggagrggcctggggstgtgggrtgscccatccccctccccatcacctttggtgggggsagggcctg

SEQ ID NO: 8 - protein, 646 aa, Homo sapiens
MAEKALEAVGCGLGPGAVAMAVTLEDGAEPPVLTTHLKKVENHITEAQRFSHLPKRSAVDIEFVELSYSVREGPCWRKRGYKTLLKCLSGKFCRRELIGIMGPSGAGRSTFMNILAGYRESGMKGQILVNGRPRELRTFRKMSCYIMQDDMLLPHLTVLEAMMVSANLKLSEKQEVKKELVTEILTALGLMSCSRTRTALLSGGQRKRLAIALELVNNPPVMFFNEPTSGLDSASCFQVVSLMKSLAQGGRTIICTIHQPSAKLFEMFDKLYILSQGQCIFKGVVTNLIPYLKGLGLHCPTYHNPADFIIEVASGEYGDLNPMLFRAVQNGLCAMAEKKSSPEKNEVPAPCPPCPPEVDPIESHTFATSTLTQFCILFKRTFLSILRDTVLTHLRFMSHVVIGVLIGLLYLHIGDDASKVFNNTGCLFFSMLFLMFAALMPTVLTFPLEMAVFMREHLNYWYSLKAYYLAKTMADVPFQVVCPVVYCSIVYWMTGQPAETSRFLLFSALATATALVAQSLGLLIGAASNSLQVATFVGPVTAIPVLLFSGFFVSFKTIPTYLQWSSYLSYVRYGFEGVILTIYGMERGDLTCLEERCPFREPQSILRALDVEDAKLYMDFLVLGIFFLALRLLAYLVLRYRVKSER

SEQ ID NO: 9 - protein, 14 aa, Artificial Sequence
  Subcloning fragment
IEAGKAESRHDKIH

(19) United States
(12) Patent Application Publication
(10) Pub. No.: US 2002/0192821 A1
(43) Pub. Date: Dec. 19, 2002

(54) Increased functional activity and/or expression of ABC transporters protects against the loss of dopamine neurons associated with Parkinson's disease

(75) Inventors: Peter B. Reiner, Vancouver (CA); Josee Roy, Vancouver (CA); Bruce P. Connop, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Active Pass Pharmaceuticals, Inc., Vancouver (CA)

(21) Appl. No.: 10/154,452

(22) Filed: May 22, 2002

Related U.S. Application Data
(60) Provisional application No. 60/327,396, filed on Oct. 4, 2001.
(60) Provisional application No. 60/292,844, filed on May 22, 2001.

(51) Int. Cl. A61K 48/00; C12N 15/85

(52) U.S. Cl. 435/455; 514/44

(57) ABSTRACT

Methods and compositions are provided for reducing the level of a catecholamine, in particular dopamine, and conjugates thereof, thus reducing catecholaminergic cell toxicity, by increasing a functional activity or increasing expression of ABC transporter polypeptides. ABC transporters serve to extrude dopamine and dopamine conjugates out of the neuron, thus preventing or reducing dopamine-associated toxicity, including cell death. Agents that increase a level of expression, or increase a functional activity, or increase both, of the ABC transporters find utility in preventing or alleviating Parkinson's disease.

### BACKGROUND OF THE INVENTION

1. Field of the Invention

The invention relates to compositions and methods useful for treating conditions associated with catecholarninergic cell toxicity. More specifically, the invention relates to treatment of Parkinson's disease and other neurodegenerative disorders by altering the functional activity or altering the expression of an ABC transporter polypeptide, which alters the intracellular level of catecholamines, to protect against the loss of doparninergic neurons, thereby preventing or alleviating Parkinson's disease.

2. Description of the Related Art

Parkinson's disease (PD) constitutes the second most common neurodegenerative disorder after Alzheimer's disease, affecting approximately 0.5% of the population over the age of 50 years (reviewed in Drukarch B. et al., Biochem. Pharmacol. 59:1023-31 (2000)). Clinical symptoms of PD include resting tremor, bradykinesia (or slowness), muscle rigidity, postural instability, and eventual slowing of mental processes and dementia. At a cellular level, PD is characterized by the progressive loss of dopaminergic neurons in the substantia nigra pars compacta and by the presence of Lewy bodies (eosinophilic intracytoplasmic inclusions) in the affected neurons. Both familial and sporadic cases of the disease occur. Approximately 5-10% of all reported cases are familial with a typical autosomal-dominant pattern of inheritance although families presenting an autosomal recessive form have also been described. Apart from an earlier symptomatic onset, dominant familial PD closely resembles the pathological and clinical features of the sporadic form of the disease.

The currently available pharmacotherapy includes “classical” dopaminomimetics, such as the dopamine precursor L-Dopa, administered with or without DA-D2 receptor agonists. Dopaminomimetics ameliorate symptoms of PD but do not halt or slow progression of the disease process that underlies PD, and may become less efficacious and cause disabling side effects with continued administration (Drukarch et al., Biochemical Pharmacology 59:1023-31 (2000)).

A need in the art exists for methods and compositions that slow or prevent the degeneration of dopaminergic neurons and loss of motor functions associated with Parkinson's disease. The present invention fulfills these needs and further provides other related advantages.

### BRIEF SUMMARY OF THE INVENTION

Briefly stated, the present invention provides methods and compositions for In one aspect, the present invention provides a method for modulating an intracellular level of a catecholamine, or a conjugate thereof, in a cell, comprising modulating a functional activity of at least one ABC transporter polypeptide of the cell, and thereby modulating the intracellular level of the catecholamine, or a conjugate thereof, in the cell. In another embodiment, the invention provides a method for modulating an intracellular level of a catecholamine, or a conjugate thereof, in a cell, comprising modulating a level of expression of at least one ABC transporter polypeptide of the cell, and thereby modulating the intracellular level of the catecholamine, or a conjugate thereof, in the cell.

In another embodiment the invention provides a method for reducing catecholaminergic cell toxicity associated with the presence of a catecholamine or a conjugate thereof in a cell, said method comprising modulating a functional activity of at least one ABC transporter polypeptide in the cell, and thereby reducing catecholaminergic cell toxicity. The invention also provides a method for reducing catecholaminergic cell toxicity associated with the presence of a catecholamine or a conjugate thereof in a cell, said method comprising modulating a level of expression of at least one ABC transporter polypeptide in the cell, thereby reducing catecholaminergic cell toxicity. In certain embodiments, the catecholamine is dopamine.

In certain embodiments, the ABC transporter polypeptide is an ABCC5 transporter polypeptide an in certain embodiments, the ABC transporter polypeptide is an ABCG4 transporter polypeptide. In other certain embodiments, the level of expression of the ABC transporter polypeptide is increased. In a further embodiment, the level of expression of the ABC transporter polypeptide is increased by transfecting or transforming the cell with a recombinant nucleic acid construct comprising a polynucleotide encoding the ABC transporter polypeptide, and in certain specific embodiments the recombinant nucleic acid construct comprises a nucleotide sequence having at least 80% identity with a sequence as set forth in any one of the sequences selected from the group consisting of SEQ ID NO:1 and SEQ ID NO:3., and the recombinant nucleic acid construct further comprises a promoter operably linked to the nucleotide sequence. In another embodiment, modulating the level of expression of the ABC transporter polypeptide comprises altering degradation of the ABC transporter polypeptide.

In certain other embodiments of the invention, the functional activity of at least one ABC transporter polypeptide is increased. In certain specific embodiments, the functional activity comprises transport or translocation transport or translocation of a substrate across a cell membrane, and transport of a substrate out of the cell, wherein the substrate comprises a catecholamine, or a conjugate thereof, or comprises dopamine, or a conjugate thereof. In other embodiments, the functional activity of the ABC transporter polypeptide comprises ATP hydrolysis.

In certain embodiments of the invention, the cell is a mammalian cell, and in certain other embodiments the cell is a catecholaminergic cell. In other embodiments, the cell is a neuronal cell.

The invention also provides a method for alleviating symptoms of a disorder associated with catecholaminergic cell toxicity in a subject, wherein the method comprises administering to the subject in need thereof at least one agent that modulates a functional activity of an ABC transporter polypeptide. In another embodiment, the invention provides a method for alleviating symptoms of a disorder associated with catecholaminergic cell toxicity in a subject, wherein the method comprises administering to the subject in need thereof at least one agent that modulates a level of expression of an ABC transporter polypeptide. In certain embodiments, the ABC transporter polypeptide is selected from the group consisting of an ABCC5 transporter and an ABCG4 transporter. In other specific embodiments the disorder is Parkinson's disease, Lewy Body dementia, spinocerebellar ataxia, Brunner syndrome, an adrenal gland disorder, schizophrenia, Tourette's syndrome, attention deficit disorder, alcoholism, drug addiction, Kelley-Seegmiller syndrome, or Lesch-Nyhan syndrome. In certain embodiments, the functional activity of the ABC transporter polypeptide is increased. In certain specific embodiments, the functional activity comprises transport or translocation transport or translocation of a substrate across a cell membrane, and transport of a substrate out of the cell, wherein the substrate comprises a catecholamine, or a conjugate thereof, or comprises dopamine, or a conjugate thereof. In other embodiments, the functional activity of the ABC transporter polypeptide comprises ATP hydrolysis. In another embodiment, the level of expression of the ABC transporter polypeptide is increased, and the increase may comprise altering degradation of the ABC transporter polypeptide.

Also provided by the invention is a non-human transgenic animal for identifying an agent that modulates catecholaminergic cell toxicity in a catecholaminergic cell. The invention provides a non-human transgenic animal that overexpresses an ABCG4 transporter polypeptide and provides a non-human transgenic animal that is a knockout animal lacking a gene encoding an ABCG4 transporter polypeptide.

In one embodiment of the invention, a method is provided for identifying an agent that is capable of modulating catecholaminergic cell toxicity in a cell of a non-human animal, comprising treating at least one first non-human animal having a disorder associated with catecholaminergic cell toxicity with a candidate agent; measuring the level of catecholaminergic cell toxicity in at least one first catecholaminergic cell of the first animal; and comparing the level of catecholaminergic cell toxicity in the first animal with the level of catecholaminergic cell toxicity in at least one second catecholaminergic cell of at least one second non-human animal having a disorder associated with catecholaminergic cell toxicity, wherein the second animal was not treated with the candidate agent, and wherein a reduction in the level of catecholaminergic cell toxicity in the first catecholaminergic cell of the first animal compared with the level of catecholaminergic cell toxicity in the second catecholaminergic cell of the second animal indicates that the agent modulates catecholaminergic cell toxicity. The invention further provides that the level of catecholaminergic cell toxicity is measured by a method comprising detecting extrusion of a catecholamine, or a conjugate thereof, from at least one first neuronal cell of the first animal; and comparing a level of catecholamine, or a conjugate thereof, extruded from the first neuronal cell with a level of catecholamine, or a conjugate thereof, from at least one second neuronal cell of the second animal that was not treated with the candidate agent, wherein an increased level of catecholamine, or a conjugate thereof, extruded from the first neuronal cell of the first animal treated with the agent compared with the level of catecholamine or a conjugate thereof extruded from the second neuronal cell of the second animal that was not treated with the candidate agent indicates that the agent modulates the toxicity of catecholamine or a conjugate thereof in the first neuronal cell. In certain embodiments, the catecholamine is dopamine, and in other embodiments, the non-human animal is a transgenic animal.

The invention further provides a method for identifying an agent that is capable of modulating expression of an ABCG4 transporter polypeptide, comprising (a) contacting a candidate agent and a first biological sample comprising at least one first cell that is capable of expressing the ABCG4 transporter polypeptide, under conditions and for a time sufficient to detect ABCG4 transporter expression; and (b) comparing a level of ABCG4 transporter expression in the first cell with a level of ABCG4 transporter expression in at least one second cell in a control sample that has not been contacted with the candidate agent, wherein an increased level of ABCG4 transporter expression in the presence of the candidate agent relative to the level of ABCG4 transporter expression in the second cell in the control sample that has not been contacted with the candidate agent indicates that the agent is capable of modulating ABCG4 transporter expression.

In another embodiment, the invention provides a method for identifying an agent that is capable of modulating transcription or expression of an ABC transporter gene, wherein modulating transcription or expression of the ABC transporter gene modulates catecholaminergic cell toxicity in a cell, comprising contacting (i) a candidate agent; (ii) a first biological sample comprising at least one first cell; and (iii) a recombinant nucleic acid construct comprising a nucleotide sequence that encodes an ABC transporter polypeptide and a promoter that is operably linked to a reporter gene, under conditions and for a time sufficient to detect transcription or expression of the reporter gene; (b) comparing a level of reporter gene transcription or expression in the first cell with a level of reporter gene transcription or expression in a second cell in a control sample that has not been contacted with the candidate agent, wherein an increased level of reporter gene transcription or expression in the first cell in the presence of the candidate agent relative to the level of reporter gene transcription or expression in the second cell in the control sample that has not been contacted with the candidate agent indicates that the candidate agent is capable of modulating ABC transporter transcription or expression, thereby modulating catecholaminergic cell toxicity in the cell. In certain embodiments, the ABC transporter gene is an ABCG4 gene and the recombinant nucleic acid construct comprises a nucleotide sequence having at least 80% identity with a sequence as set forth in SEQ ID NO:3. In another embodiment, the ABC transporter gene is an ABCC5 gene and the recombinant nucleic acid construct comprises a nucleotide sequence having at least 80% identity with a sequence as set forth in SEQ ID NO:1. In other certain embodiments, the reporter gene is selected from the group consisting of chloramphenicol acetyltransferase, firefly luciferase, beta-galactosidase, and green fluorescent protein.

The invention provides a method for identifying an agent that is capable of modulating catecholaminergic cell toxicity, comprising (a) contacting (i) a candidate agent and (ii) a first biological sample comprising at least one first cell, under conditions and for a time sufficient to detect catecholaminergic cell toxicity in the first cell, wherein the first cell is capable of expressing at least one ABC transporter polypeptide; (b) comparing a level of catecholaminergic cell toxicity in the first cell with a level of catecholaminergic cell toxicity in at least one second cell in a control sample that has not been contacted with the candidate agent, wherein a reduced level of catecholaminergic cell toxicity in the first cell relative to the level of catecholaminergic cell toxicity in the second cell in the control sample that has not been contacted with the candidate agent indicates that the agent is capable of modulating catecholaminergic cell toxicity. In certain embodiments the ABC transporter polypeptide is selected from the group of an ABCC5 transporter polypeptide and an ABCG4 transporter polypeptide. In other embodiments, modulating catecholaminergic cell toxicity comprises modulating expression of the ABC transporter polypeptide and in specific embodiments, expression of the ABC transporter polypeptide is increased. In other embodiments, modulating catecholaminergic cell toxicity comprises modulating a functional activity of the ABC transporter polypeptide, and in particular embodiments, the functional activity of the ABC transporter polypeptide is increased. In other certain embodiments, the functional activity of the ABC transporter polypeptide comprises modulating transport or translocation of a substrate across a membrane of the first cell, and in other embodiments the functional activity of the ABC transporter polypeptide comprises modulating extrusion of a substrate from the first cell. In specific embodiments, the substrate comprises a catecholamine, or a conjugate thereof, and in other embodiments, the substrate comprises dopamine, or a conjugate thereof. The invention further provides that the functional activity of the ABC transporter polypeptide comprises ATP hydrolysis and that modulating expression of the ABC transporter comprises altering degradation of the ABC transporter polypeptide.

These and other embodiments of the present invention will become apparent upon reference to the following detailed description and attached drawings. All references disclosed herein are hereby incorporated by reference in their entireties as if each was incorporated separately.

### BRIEF DESCRIPTION OF THE DRAWINGS

FIG. 1 depicts the effect of ABC transporter polypeptide expression on intracellular dopamine-induced toxicity. hDAT cells were transiently transfected with (1) pCEP4 empty construct; (2) ABCC5 transporter expression vector; (3) ABCG4 transporter expression vector; (4) ABCx transporter expression vector; (5) ABCy transporter expression vector, and (6) a non-functional transmembrane ABC transporter, ABCz. Dopamine-induced cytotoxicity was assessed by a methylthiazoletetrazolium (MTT) assay (Sigma-Aldrich, St. Louis, Mo.). Data were combined from 1-3 experiments (n=4-14). The symbol “*” represents cell viability significantly different from pCEP4 empty construct treatment group. *p<0.05 and ***p<0.001.

FIG. 2 shows the effect of ABC transporter inhibitors glipizide (FIG. 2A) and probenecid (FIG. 2B) on intracellular dopamine-induced toxicity. hDAT cells were transiently transfected with the pCEP4 empty construct or an expression vector encoding one of the ABC transporters ABCC5 or ABCG4. Data represent one experimental trial with n=2. Data for each ABC transporter are presented as the percent of the MTT activity relative to each individual vector control group (“No treatment” group).

FIG. 3 shows the effect of ABCC5 and ABCG4 transporter polypeptides and ABCC5 and ABCG4 mutant polypeptides on dopamine-cytotoxicity. hDAT cells were transiently transfected with the pCEP4 empty construct or an expression vector encoding ABCC5 transporter, ABCG4 transporter, an ABCC5 mutant, or an ABCG4 mutant. Data were combined from 2 different experiments (n=5-9). The symbol “*” represents cell viability significantly different from the pCEP4 empty construct treatment group. ***p<0.001.

### DETAILED DESCRIPTION OF THE INVENTION

The invention provides compositions and methods for decreasing catecholamine-induced toxicity in catecholaminergic cells. More specifically, the invention provides compositions and methods for reducing dopamine-induced toxicity in catecholaminergic cells of the central nervous system. The importance and clinical relevance of this discovery are best understood with reference to the background studies of catecholamine regulation, particularly, dopamine regulation, as discussed below.

A subset of dominant PD cases (PARK1) has been linked to chromosome 4q21-23 and associated with mutations in the gene encoding the α-synuclein protein (Polymeropoulos et al., Science 276:2045-47 (1997)). Although little is known about the function of α-synuclein, studies of its avian orthologue, the synelfin gene product, suggest that it may play a role in neuronal plasticity and learning mechanisms (George et al., Neuron 15:361-72 (1995)). Two missense mutations of the gene have been identified: Ala53Thr, in families of the Central Mediterranean region (Polymeropoulos et al., supra) and Ala30Pro in German families (Krüger et al., Nat. Genet. 18:106-8 (1998)). α-Synuclein is a pre-synaptic protein abundantly expressed in various regions of the brain including the substantia nigra pars compacta. oc-synuclein shares both physical and functional homology with the 14-3-3 family of proteins that possess “chaperoning” activity (Ostrerova et al., J. Neurosci. 19:5782-91 (1999)).

Mice lacking α-synuclein display impaired dopaminergic function under certain stresses, which has led to the suggestion that α-synuclein is a pre-synaptic chaperone protein that may represent a regulator of dopamine release (Abeliovich A. et al., Neuron 25:239-52 (2000)). The physiochemical properties of α-synuclein indicate that it is a natively unfolded molecule that can self-aggregate and form fibrils in vitro (Weinreb et al., Biochemistry 35:13709-15 (1996); Giasson et al., J. Biol. Chem. 276:2380-86 (2001)). For example, in studies using recombinant proteins, mutated forms of α-synuclein were more prone to self-aggregation under certain conditions and stresses when compared to the wild-type protein (Ostrerova-Golts N. et al., J. Neurosci. 20:6048-54 (2000)).

Ubiquitin and α-synuclein represent major components of Lewy bodies that are found in both sporadic and familial PD patients, suggesting that abnormal aggregation of α-synuclein or ubiquitin or both may play a central role in PD pathogenesis (Spillantini M. G. et al., Nature 388:839-40 (1997)). This hypothesis is supported by data that showed over-expression of the wild-type human α-synuclein protein in mice (Masliah et al., Science 287:1265-69 (2000)) or wild-type or mutated variants in Drosophila (Feany et al., Nature 404:394-98 (2000)) triggered focal accumulation of α-synuclein and the relatively selective degeneration of dopaminergic neurons.

Certain juvenile forms of familial PD (AR-JP, PARK2) are also characterized by an autosomal-recessive inheritance. An AR-JP locus has been identified on chromosome 6q25.2-q27 and designated as the gene parkin (Kitada T. et al., Nature 392:605-8 (1998)). Missense mutations and homozygous deletions have been found in 7 of the 12 coding exons of the parkin gene (Abbas N. et al., Hum. Mol. Genet. 8:567-74 (1999)). The polypeptide product of theparkin gene shares some sequential similarity with the ubiquitin family of proteins (Kitada T. et al., Nature 392:605-8 (1998)) and has recently been identified as an ubiquitin-protein ligase (Shimura H. et al., Nature 25:302-5 (2000)). Thus, parkin may be involved in proteosomal proteolytic processing. AR-JP has a mean age of onset of ˜28 years, and the pathology of the disease indicates a slower progression of disease phenotype and no apparent formation of Lewy bodies. At present, only a fraction of reported PD cases are believed to be associated with mutations in either the α-synuclein or the parkin genes, which suggests that the pathogenesis of this disease is multifactorial and may involve both genetic and environmental factors.

Epidemiological studies have suggested that pesticide or herbicide exposure or exposure to both is associated with an increased risk of developing PD (Menegon et al., Lancet 352:1344-46 (1998)). In humans, the pro-toxin N-methyl-4-phenyl-1,2,3,6-tetrahydropyridine (MPTP) produces an acute parkinsonian syndrome that is virtually indistinguishable from idiopathic PD (Langston et al., Science 219:979-80 (1983)). Data from animal studies indicates that the MPTP metabolite, 1-methyl-4-pyridinium (MPP+), acts as a mitochondrial poison that is selectively imported into dopaminergic neurons due to its preferential affinity for the dopamine uptake transporter (DAT) (Nicklas et al., Life Sci. 36:2503-08 (1985), Tipton. et al., J. Neurochem. 61:1191-06 (1993), Tolwani et al., Lab. Anim. Sci. 49:363-71 (1999)). Following recognition of MPTP's toxicity and mode of action, several groups reported identifying a selective defect in mitochondrial complex I activity in PD patients (Parker et al., Ann. Neurol. 26:719-23 (1989)).

In one study, chronic systemic administration of the common pesticide rotenone caused a pathology in rats that resembled human PD (Betarbet et al., Nature 3:1301-05 (2000)). Rotenone also caused an inhibition of mitochondrial complex I activity (Betarbet et al., supra). In view of these findings, genetic polymorphisms may possibly affect the activity of detoxification mechanisms that modulate susceptibility to environmental toxins such as rotenone, thus playing a role in PD pathogenesis. For example, homozygosity for a deletion in the gene encoding specific isoforms of glutathione-S-transferase (GST) correlates with an increased risk of developing PD (Stroombergen et al., Hum. Exp. Toxicol. 18:141-45 (1999)). Perturbation of detoxification enzymes such as GST may also impair the ability of a cell to cope with the level of free radicals and reactive oxygen species (ROS) generated through normal cellular metabolism.

Reactive oxygen species and oxidative stress may contribute to the pathogenesis of PD because dopamine can be oxidized to yield both highly reactive DA-metabolites and other ROS. Postmortem studies of PD substantia nigra have shown that within the substantia nigra, indices of oxidative stress were increased, including decreased mitochondrial activity, increased iron levels, reduced GSH content, and oxidative damage to lipids, proteins, and DNA (reviewed in Jenner et al., Neurology 47:161-70 (1996)).

Processing (or recycling) of the neurotransmitter dopamine (DA) may represent an important source of intracellular free radical production mediated either through enzymatic oxidation or by cycling/autooxidation reactions (reviewed in Drukarch et al., Biochem. Pharmacol. 59:1023-31 (2000); Zhang et al., J. Neurochem. 74:970-78 (2000)). Upon stimulation, dopaminergic neurons release the neurotransmitter DA at the synaptic junction where it can react with dopamine receptors on the pre- or post-synaptic neuronal membrane. Following synaptic DA release, termination of dopaminergic transmission is regulated by (1) the synaptic enzymatic metabolism of DA by two enzymes, monoamine oxidase (MAO) and catechol-o-methyl transferase (COMT), which yields 3,4-dihydroxyphenylacetic acid (DOPAC) and homovanillic acid (HVA); and by (2) DA uptake mechanisms that involve selective reuptake of DA into dopaminergic neurons through the dopamine uptake transporter (DAT). This recycled cytoplasmic DA, or even newly synthesized DA, may then undergo one of the following reactions: (1) degradation by the enzyme monoamine oxidase (MAO) to yield H₂O₂ and DOPAC; (2) spontaneous “cycling” in autooxidation reactions, leading to the formation of DA-quinone molecules; or (3) conversion to DA-semiquinone by the enzymatic activity of the NADPH-cytochrome P450 reductase system.

While intracellular DA metabolites such as DOPAC are extruded from the cell, the DA-quinone and semiquinone molecular species may polymerize and lead to the formation of neuromelanin, the pigment that in adult humans lends the substantia nigra its characteristic dark color. Iron may also be present in neuromelanin granules. Reactive iron ions in the presence of H₂O₂ or free DA could promote the formation of the highly reactive OH. radicals through the Fenton reaction. This could consequently facilitate DA-autooxidation (Good et al., Brain Res. 593:343-46 (1992)). DA autooxidation and accumulation of neuromelanin do not appear to occur at the same rate in all DA neurons; however, susceptible populations of dopaminergic neurons to PD pathology correspond to those cells heavily loaded with neuromelanin granules (Hirsch E. et al., Nature 334:345-48 (1988)). Consequently, regulation of DA-quinone/semiquinone formation and neuromelanin aggregation may also be important in the development of PD. Normally, these highly reactive DA-quinone and semiquinone by-products of DA degradation can be detoxified in cells by the glutathione (GSH) conjugation pathway mediated by the enzyme glutathione-S-transferase (GST) and, more specifically for DA molecules, by the GSTM2-2 isoform (Segura-Aguilar J. et al., J. Biol. Chem. 272:5727-31 (1996)). GST enzymes are sensitive to downregulation by GSH conjugates such as GSH-conjugated DA (Ploemen J. et al., Chem.-Biol Interact. 90:87-99 (1994)), which suggests that increased buildup of cellular GSH-conjugated DA-quinone (DA-Q-GSH) metabolites could feedback in a negative manner to decrease the normally protective function of the GST enzyme. Thus, although GST enzymes such as GSTM2-2 will “detoxify” reactive DA-quinone and semiquinone species, accumulation of DA-Q-GSH within the cell may feedback to inhibit this protective process. Reduction of the protective activity of the GST enzyme may lead to a generalized build up of toxic DA metabolites, as well as other free radicals.

If such a build-up occurs, mitochondria may be the first organelle affected when the ability of the cell to deal with free radicals is impaired. Mitochondria represent the major site of free radical production through normal metabolism/ATP production (reviewed in Olanow et al., Ann. Rev. Neurosci. 22:123-44 (1999)). Mitochondrial defects may, in turn, lead to increased calcium influx into the cells, activation of nitric oxide synthetase (NOS), and subsequent increases in nitric oxide (NO) production. Possibly, NO may displace iron from its binding site on the ferritin protein, increasing the level of reactive iron, and may also alter tyrosine residues present on various proteins, which would exacerbate this toxic cycle. Furthermore, the presence of NO-modified proteins (Good et al., J. Neuropathol. Exp. Neurol. 57:338 (1998)) and nitrated α-synuclein proteins (Giasson et al., Science 290:985-89 (2000)) has been observed in the core of Lewy bodies in PD patients.

DA is able either to directly or indirectly induce oxidative stress when added exogenously to various cell types (see Stokes et al., J. Neurosci. Res. 55:659-65 (1999); Drukarch et al., Biochem. Pharmacol. 59:1023-31 (2000)). The link between DA-induced oxidative stress and mutations in α-synuclein associated with familial PD has been examined. Familial-linked mutations in α-synuclein either may render cells more susceptible to DA (or to other toxic insults) or may increase the propensity of α-synuclein to aggregate into Lewy body-like inclusions, particularly in the presence of DA or iron or both (Ko et al., J. Neurochem. 75:2546-54 (2000); Ostrerova-Golts et al., J. Neurosci. 20:6048-54 (2000); Tabrizi et al., Hum. Mol. Genet. 9:2683-89 (2000); Lee et al., FASEB J. 15:919-26 (2001); Lee et al., J. Neurochem. 76:998-09 (2001)). From these studies, oxidative stress appears to be induced by DA, and in conjunction with reactive iron, DA may increase the ability of α-synuclein to aggregate, rendering dopaminergic neurons more susceptible to degeneration observed in PD.

The present invention is directed to methods and compositions for slowing or preventing the degeneration of dopaminergic neurons and the loss of motor functions associated with PD. More specifically, the present invention relates to the observation that the presence of an ABC transporter polypeptide in a neuronal cell results in reduced cytotoxicity. Not wishing to be bound by theory, preventing or reducing catecholaminergic cell toxicity may be accomplished by promoting the extrusion of catecholamines and conjugates thereof, such as DA and any conjugated DA-Q-GSH complex from dopaminergic cells to release GST from potential feedback inhibition by the DA or DA conjugates.

The present invention relates to the discovery that by modulating a function or expression or both of an ABC transporter polypeptide, the intracellular level of a catecholamine or a catecholamine conjugate, such as dopamine or a conjugate thereof, can be modulated. More specifically, increasing a functional activity or increasing expression (thereby increasing the number of functional ABC transporter polypeptide molecules in the cell) an ABC transporter reduces catecholaminergic cell toxicity. As described in greater detail herein, this observation may be exploited according to certain embodiments of the present invention to treat a disorder associated with catecholaminergic cell toxicity, for example, Parkinson's disease.

In one embodiment of the invention, a method is provided for modulating or altering (increasing or decreasing in a statistically significant manner relative to an appropriate control) the intracellular level of a catecholamine, or a conjugate thereof, in a cell, comprising modulating a functional activity of at least one ABC transporter polypeptide of the cell. In another embodiment of the invention, a method is provided for modulating (increasing or decreasing in a statistically significant manner) the intracellular level of a catecholamine comprising modulating, preferably increasing, the level of expression of an ABC transporter.

Catecholamines form a general class of ortho-dihydroxyphenylalkylamines derived from tyrosine. Catecholamines include epinephrine (adrenaline), norepinephrine (noradrenaline), and dopamine, which act as hormones or neurotransmitters in cells. A catecholaminergic cell, as referred to herein, is one in which one or more catecholamines may be used as a hormone or neurotransmitter. For example, adrenal cells (particularly those of the adrenal medulla) employ epinephrine as the predominant neurohormone. Epinephrine and norepinephrine also may act as neurotransmitters in neurons, cells of the nervous system. Neuronal cells that use dopamine as the primary neurotransmitter are catecholaminergic cells referred to as dopaminergic cells.

Catecholamine conjugates form during catabolism of catecholamines and include, but are not limited to, glutathione conjugates of catecholamine quinones and semiquinones origin. In addition to normal catabolism of catecholamines that proceeds in part through various enzymatic pathways (monoaminooxidase, catechol-o-methyltransferase, and phenolsulphotransferase), nonenzymatic oxidative pathways may also take place. The latter pathway is also referred to as autooxidation and is a complex process involving a large number of intermediates (Drukarch et al., Biochem. Pharmacol. 59:1023-31 (2000)). Catecholamine conjugates include dopamine conjugates. Autooxidation of the catecholamine dopamine (DA) yields a product called DA-quinone. Similar to catecholamine-quinones, DA-derived quinones are generally highly reactive, electron-deficient molecules that may covalently bind to cellular nucleophiles (e.g., DNA) and to reduced sulfhydryl groups contained in protein cysteinyl residues and the thiol antioxidant glutathione (GSH). GSH-conjugated forms of DA-quinones and GSH-conjugated forms of semiquinones (formed by conversion of DA by the NADPH-cytochrome P450 reductase system, discussed herein) present in a catecholaminergic cell likely contribute to oxidative stress and oxidative damage that contribute to catecholaminergic cell toxicity.

As noted above, according to certain embodiments of the invention, modulating the intracellular level of a catecholamine or a conjugate thereof, comprises modulating a functional activity or a level of expression of an ABC transporter, or both. ABC (ATP binding cassette) transporter polypeptides represent a large superfamily of proteins with conserved features in both prokaryotes and eukaryotes. ABC transporters catalyze ATP-dependent transport or translocation of endogenous or exogenous substrates across extracellular and intracellular membranes (Borst, Seminar in Cancer Biology 8:131-213 (1997)). ABC transporters exist in many living systems, including bacteria, yeast, and mammals. Genetic variations in ABC transporter genes cause or contribute to a number of human disorders, for example, cystic fibrosis, neurological disease, retinal degeneration, and cholesterol transport defects. ABC transporters are also associated with multidrug resistance in cells by virtue of the capability of certain ABC transporters to extrude cytotoxic anti-cancer drugs out of the cell (see, e.g., review of Dean et al., Genome Res. 11:1156-66 (2001)). ABC transporters are characterized by having an ATP binding cassette that contains two conserved peptide motifs, Walker A and Walker B, both of which are involved in ATP binding. A third conserved region, the ABC signature motif, forms part of the ATP binding cassette and is located between the Walker A and Walker B motifs (Dean et al., supra). A functional ABC transporter typically has two transmembrane domains that contain 6-11 membrane-spanning α-helices. ABC transporter genes are organized as either full transporters, containing two transmembrane domains and two ATP binding cassettes, or as half transporters. The latter are believed to form either homodimers or heterodimers to become a functional transporter. The 48 known human transporters are further classified into seven sub-families on the basis of presently known gene and protein structural information (Dean et al., supra). The ABC transporter polypeptides contemplated by the present invention are preferably of mammalian origin, and more preferably are of human origin.

According to the present invention, to modulate the intracellular level of a catecholamine or of dopamine, or of their respective conjugates, a functional activity of, or a level of expression of, or both a functional activity and expression of any of the 48 known transporters may be modulated. The ABC transporter useful in the present invention may include any of the 13 transporters in the ABCA family (ABCA1-13); any of the 11 ABC transporters in the ABCB family (ABCB1-ABCB11); any of the 12 ABC transporters in the ABCC family (ABCC1-ABCC12); any of the 4 transporters in the ABCD family (ABCD1-ABCD4); any ABCE1 ABC transporter; any of the 3 transporters in the ABCF family (ABCF1-ABCF3); and any of the five ABC transporters in the ABCG family (ABCG1, ABCG2, ABCG4, ABCG5, and ABCG8). In certain embodiments the ABC transporter polypeptide may include ABCA2, ABCB1, and ABCB9 that are expressed in the brain, or may include transporters that are expressed ubiquitously, for example, ABCA1, ABCB5, ABCC5, ABCF1, ABCF2, ABCF3, and ABCF4, or may include ABCA2, ABCB1, ABCC2, ABCC3, and ABCG2, which are involved in transport of toxic molecules out of the cell (Dean et al., supra), or may include ABCA2, ABCB1, and ABCB9, which are ABC transporter polypeptides that may be expressed in a catecholaminergic cell (expressed in the adrenal gland or brain, Dean et al., supra). In a highly preferred embodiment the ABC transporter includes ABCC5 and ABCG4.

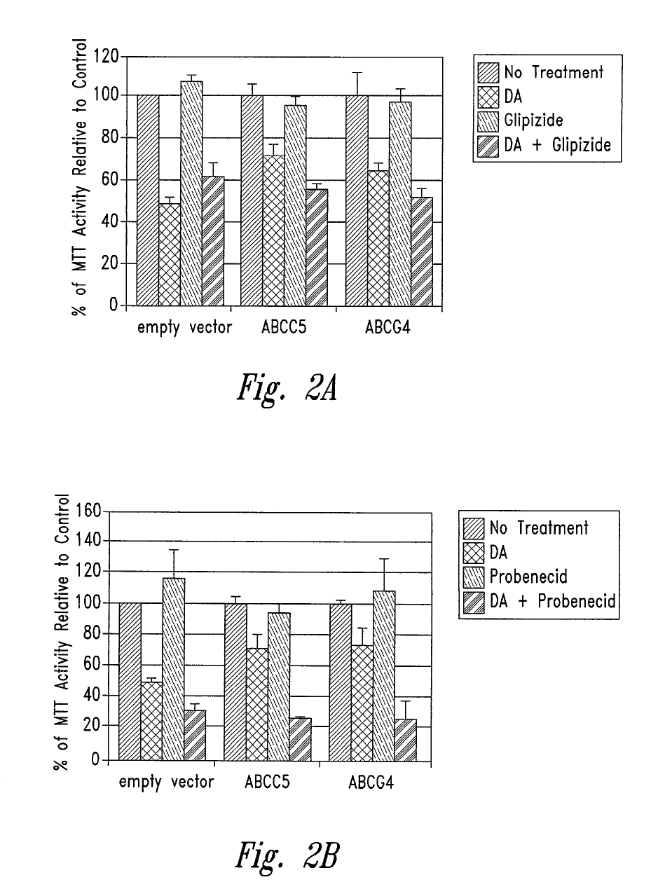

In a preferred embodiment, catecholaminergic cell toxicity associated with the intracellular presence of a catecholamine or a conjugate thereof, wherein the catecholamine may be dopamine, is reduced according to a method that modulates a functional activity and/or expression of at least one ABC transporter. An ABC transporter useful for practicing this invention may be identified in an in vitro assay, such as a cell-based assay that represents a model system for analyzing catecholaminergic cell toxicity. By way of example, the cell line hDAT-SK-N-MC (hDAT), a human neuroblastoma cell line with catecholaminergic properties, stably expresses human dopamine transporter (hDAT) cDNA. hDAT transports dopamine into the cell, producing a measurable degree of DA toxicity, which is mediated through the intracellular accumulation of DA. hDAT cells were transiently transfected with expression vectors encoding one of a number of functional or inactive ABC transporter polypeptides (see Example 1). ABCC5 and ABCG4 transporters conferred protection against dopamine-induced toxicity. Inhibitors of ABC transporters, such as glipizide and probenecid, reversed the protective effect of ABCC5 and ABCG4, as shown in FIG. 2. Not wishing to be bound by a specific mechanism, the data suggest that these transporters may be functioning as “pumps” to transfer dopamine or dopamine-conjugates or both out of the cells.

In a preferred embodiment of the invention, a functional activity of at least one ABC transporter polypeptide is increased. As used interchangeably herein, “ABC transporter activity,” “biological activity of an ABC transporter,” or “functional activity of an ABC transporter,” refers to an activity exerted by an ABC transporter protein, polypeptide, or nucleic acid molecule on an ABC transporter-responsive cell or on an ABC transporter polypeptide substrate, as determined in vivo or in vitro, according to standard techniques. In a preferred embodiment, the functional activity comprises the transport of a substrate across a cell membrane or transport of a substrate out of a catecholaminergic cell, wherein the substrate comprises a catecholamine or a conjugate thereof. In a highly preferred embodiment of the invention, the catecholamine is dopamine.

In another preferred embodiment of the invention, the level of expression of an ABC transporter polypeptide is increased (i.e., statistically significant increase relative to an appropriate control). In a further embodiment of the invention, the expression of the ABC transporter polypeptide is increased by transforming or transfecting a cell with a recombinant nucleic acid construct that encodes the ABC transporter or a variant thereof. In a preferred embodiment of the invention, expression of an ABCC5 or an ABCG4 polypeptide or both is increased. The present invention also contemplates that expression may be altered by activating or deactivating a regulatory element, such as a promoter. Alternatively, a mutation or polymorphism in a gene encoding an ABC transporter or in a regulatory element that decreases expression may be corrected by replacing the mutated sequence with a wild-type sequence, or expression may be altered by inserting an antisense sequence to bind to an overexpressed sequence or to a regulatory sequence.

As used herein, the term “ABCC5 transporter polypeptide” refers to a polypeptide that comprises the sequences as provided herein (SEQ ID NO:2). See Genebank Accession Nos. AF146074 and AF104942; U.S. Pat. No. 6,162,616. The term “ABCG4 transporter polypeptide” refers to a polypeptide that comprises the sequence as provided herein in SEQ ID NO:4 (as disclosed in application Ser. No. 10/090,455 and herein incorporated by reference). The ABCC5 transporter polypeptide and the ABCG4 transporter polypeptide of the present invention are encoded by polynucleotides having the nucleotide sequences as set forth in SEQ ID NO:1 (see Genebank Accession Nos. AF146074 and AF104942; U.S. Pat. No. 6,162,616) and SEQ ID NO:3 (as disclosed in application Ser. No. 10/090,455), respectively. An “ABCC5 polynucleotide” is any polynucleotide that encodes at least a portion of an ABCC5 transporter polypeptide, or that is complementary to such a polynucleotide. Similarly, an “ABCG4 polynucleotide” is any polynucleotide that encodes at least a portion of an ABCG4 transporter polypeptide, or that is complementary to such a polynucleotide. Polynucleotides may be single-stranded (coding or antisense) or double-stranded, and may be DNA (genomic, cDNA, or synthetic) or RNA molecules. Additional coding or non-coding sequences may be, but need not be, present within a polynucleotide of the present invention, and a polynucleotide may be, but need not be, linked to other molecules and/or support materials.

Expression of the ABCC5 transporter polypeptide or the ABCG4 polypeptide may be increased by transfecting or transforming a cell with a recombinant nucleic acid construct that comprises a nucleotide sequence having at least 80% identity with a sequence as set forth in SEQ ID NO:1 or SEQ ID NO:3, respectively. More preferably, the ABCC5 and the ABCG4 polypeptides are encoded by polynucleotides having at least 82%, 85%, or 88% identity, and more preferably at least 90%, 92%, 94%, 96%, and 98% identity with the nucleotide sequences set forth in SEQ ID NOS:1 and 3, respectively. The polynucleotides useful in the present invention may differ from the nucleotide sequences shown in SEQ ID NOS: 1 and 3 because of degeneracy of the genetic code and would still encode the ABC transporter proteins encoded by the sequences set forth in SEQ ID NOS:1 and 3. The percent identity of a polynucleotide with an ABCC5 or ABCG4 transporter polynucleotide as disclosed herein may be readily determined by comparing sequences using computer algorithms well known to those having ordinary skill in the art, such as Align or the BLAST algorithm (Altschul, J. Mol. Biol. 219:555-65, 1991; Henikoff and Henikoff, Proc. Natl. Acad. Sci. USA 89:10915-19, 1992), which is available at the NCBI website (http://www/ncbi.nlm.nih.gov/cgi-bin/BLAST). Default parameters may be used.

Certain variants are substantially homologous to a native gene. Such polynucleotide variants are capable of hybridizing under moderately stringent conditions to a naturally occurring DNA or RNA sequence encoding a native ABCC5 transporter polypeptide or a native ABCG4 transporter polypeptide (or a complementary sequence). Suitable moderately stringent conditions include, for example, pre-washing in a solution of 5× SSC, 0.5% SDS, 1.0 mM EDTA (pH 8.0); hybridizing at 50° C.-70° C., 5× SSC for 1-16 hours; followed by washing once or twice at 22-65° C. for 20-40 minutes with one or more each of 2×, 0.5× and 0.2× SSC containing 0.05-0.1% SDS. For additional stringency, conditions may include a wash in 0.1× SSC and 0.1% SDS at 50-60° C. for 15 minutes. As known to those having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used, and of the blotted, proband nucleic acid sample. Accordingly, it will be appreciated that suitably stringent conditions can be readily selected without undue experimentation when a desired selectivity of the probe is identified, based on its ability to hybridize to one or more certain proband sequences while not hybridizing to certain other proband sequences.

A variant of an ABC transporter polypeptide encoded by the polynucleotide sequences that are contemplated by the invention and disclosed herein shares common structural domains, or motifs, or a common functional activity, or both with the disclosed ABCC5 and ABCG4 polypeptides. A variant of an ABCC5 transporter polypeptide or an ABCG4 transporter polypeptide, has at least 70% or 75% identity, preferably at least 80%, 82%, or 85% identity, and more preferably at least 88%, 90%, 92%, 94%, 96%, and 98% identity with the polypeptides encoded by the nucleotide sequences set forth in SEQ ID NOS:1 and 3, and which have the amino acid sequences set forth in SEQ ID NO:2 and SEQ ID NO:4, respectively.

The polynucleotides useful in the present invention may differ from the nucleotide sequences shown in SEQ ID NOS:1 and 3 in that the encoded ABC transporter polypeptides may contain conservative substitutions of an amino acid. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. A conservative substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an ABCC5 or an ABCG4 transporter protein is preferably replaced with another amino acid residue from the same side chain family. The ABC transporter polypeptides may also, or alternatively, contain nonconservative changes. Such ABCC5 and ABCG4 transporter polypeptides differ in amino acid sequence from the sequences set forth in SEQ ID NOS:2 and 4, respectively, yet retain functional activity.

Recombinant nucleic acid constructs comprising a polynucleotide sequence encoding an ABC transporter polypeptide contemplated by the present invention may be prepared using any of a variety of expression vectors according to methods described herein and known in the art. Expression may be achieved in any appropriate host cell that has been transformed or transfected with an expression vector containing a DNA molecule that encodes a recombinant ABC transporter polypeptide. A host cell can be any prokaryotic or eukaryotic cell. For example, an ABC transporter polypeptide can be expressed in bacterial cells such as E. coli, or expressed in insect cells, yeast, or mammalian cells (such as Chinese hamster ovary cells (CHO) or COS cells). Other suitable host cells are known to those skilled in the art. Preferably the host cell is a mammalian cell, more preferably a catecholaminergic cell, and more preferably a neuronal cell. Examples of mammalian expression vectors include pCDM8 (Seed, Nature 329:840(1987)) and pMT2PC (Kaufman et al., EMBO J 6:187-195 (1987)). When used in mammalian cells, the expression vector's control functions are often provided by viral regulatory elements. For other suitable expression systems for both prokaryotic and eukaryotic cells see Sambrook et al., Molecular Cloning: A Laboratory Manual. 3rd, ed., Cold Spring Harbor Laboratory, Cold Spring Harbor Laboratory Press, Cold Spring Harbor, N.Y., 2001.

In general, recombinant constructs of the subject invention will also contain elements necessary for transcription and translation. The choice of the promoter will depend upon the cell type to be transformed and the degree or type of control desired. Promoters can be constitutive or active and may further be cell type specific, tissue specific, individual cell specific, event specific, temporally specific, or inducible. Cell-type specific promoters and event type specific promoters are preferred, for example, promoters that are specific for neuronal cells. Examples of constitutive or nonspecific promoters include the SV40 early promoter (U.S. Pat. No. 5,118,627), the SV40 late promoter (U.S. Pat. No. 5,118,627), CMV early gene promoter (U.S. Pat. No. 5,168,062), and adenovirus promoter. In addition to viral promoters, cellular promoters are also amenable within the context of this invention. In particular, cellular promoters for the so-called housekeeping genes are useful. Viral promoters are preferred, because generally they are stronger promoters than cellular promoters. Promoter regions have been identified in the genes of many eukaryotes including higher eukaryotes, such that suitable promoters for use in a particular host can be readily selected by those skilled in the art.

Inducible promoters may also be used. These promoters include MMTV LTR (PCT WO 91/13160), inducible by dexamethasone; metallothionein promoter, inducible by heavy metals; and promoters with cAMP response elements, inducible by cAMP. By using an inducible promoter, the nucleic acid sequence encoding an ABC transporter polypeptide may be delivered to a cell by the subject invention expression construct and will remain quiescent until the addition of the inducer. This allows further control on the timing of production of the gene product.

Event-type specific promoters are active or up-regulated only upon the occurrence of an event, such as tumorigenicity or viral infection. The HIV LTR is a well known example of an event-specific promoter. The promoter is inactive unless the tat gene product is present, which occurs upon viral infection. Some event-type promoters are also tissue-specific.

Within certain embodiments, a polynucleotide encoding an ABC transporter may be formulated so as to permit entry into a cell of a mammal and expression therein. Such formulations are particularly useful for therapeutic purposes, as described below. Those having ordinary skill in the art will appreciate that many ways exist to achieve expression of a polynucleotide in a target cell, and any suitable method may be employed. For example, a polynucleotide may be incorporated into a viral vector using well known techniques. A viral vector may additionally transfer or incorporate a gene for a selectable marker (to aid in the identification or selection of transduced cells) and/or a targeting moiety, such as a nucleotide sequence that encodes a ligand for a receptor on a specific target cell, which renders the vector target specific. Targeting may also be accomplished using an antibody, by methods known to those having ordinary skill in the art.

The recombinant nucleic acid expression vectors also include one or more regulatory sequences operatively linked to the nucleic acid sequence to be expressed, and selected on the basis of the host cells to be used for expression. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells and those that direct expression of the nucleotide sequence only in certain host cells (e.g., tissue-specific regulatory sequences). Those skilled in the art will appreciate that the design of the expression vector can depend on such factors as the choice of the host cell to be transformed, the level of expression of protein desired, and the like. The expression vectors of the invention can be introduced into host cells to thereby produce an ABC transporter polypeptide, including fusion proteins, encoded by nucleic acids as described herein (e.g., ABCC5 and ABCG4 transporter proteins, mutant forms of the transporter proteins, fusion proteins, and the like).

The recombinant mammalian expression vectors useful for the present invention may be capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include neuron-specific promoters (e.g., the neurofilament promoter, Byrne et al., Proc. Natl. Acad. Sci. USA 86:5473-77 (1989)).

The recombinant nucleic acid construct can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al., supra, and other laboratory manuals.

For stable transfection of mammalian cells, depending upon the expression vector and transfection technique used, only a small fraction of cells may integrate the foreign DNA into their genome. To identify and select these integrants, a gene that encodes a selectable marker (e.g., resistance to antibiotics) is generally introduced into the host cells along with the gene of interest. Preferred selectable markers include those that confer resistance to drugs, such as G418, hygromycin, and methotrexate. A polynucleotide encoding a selectable marker can be introduced into a host cell on the same vector as that encoding an ABC transporter polypeptide or can be introduced on a separate vector. Cells stably transfected with the introduced polynucleotide can be identified by drug selection (e.g., cells that have incorporated the selectable marker gene will survive, while the other cells die).

In preferred embodiments, elements that increase the expression of the desired ABC transporter polypeptide are incorporated into the construct. Such elements include internal ribosome binding sites (IRES; Wang and Siddiqui, Curr. Top. MicrobioL Immunol 203:99 (1995); Ehrenfeld and Semler, Curr. Top. Microbiol. Immunol. 203:65 (1995); Rees et al., Biotechniques 20:102 (1996); Sugimoto et al., Biotechnology 12:694 (1994)). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection and the like. Protein levels may be assayed by any known method, including ELISA, western blot, immunoprecipitation, immunocytochemistry, or other well known techniques.

In another embodiment of the invention, a functional activity of an ABC transporter is modulated. A functional activity of an ABC transporter includes the ability to act as an energy-dependent (ATP) molecular pump, that is, the ABC transporter uses the energy generated by ATP hydrolysis (ATPase activity) for transport of a substrate against the concentration gradient of the substrate. As discussed herein, the ATP binding cassette is comprised of at least three peptide motifs, Walker A, Walker B, and an ABC signature motif. ATP hydrolysis in a particular cell or cell type may be increased by restoring activity to a level normally observed associated with the ABC transporter in a normal cell. Alternatively, ATP hydrolysis activity of an ABC transporter may be increased above a level normally associated with the ABC transporter. ATP hydrolysis activity may be modulated by mutagenesis of an ABC transporter polypeptide, for example, by mutagenesis of amino acids within the ATP binding cassette portion of the polypeptide, according to methods described herein and known in the art.

To modulate a functional activity of an ABC transporter polypeptide, (such as to increase ATP hydrolysis activity or to alter the affinity of the ABC transporter for a substrate or for a ligand that affects a functional activity of the ABC transporter), mutations can be introduced into an ABC transporter by standard techniques known to those having skill in the art, such as site-directed mutagenesis, ligase chain reaction mutagenesis, and PCR-mediated mutagenesis of the encoding polynucleotide sequence. In a preferred embodiment of the invention, the amino acid sequence of an ABCC5 polypeptide or an ABCG4 polypeptide may be changed by inserting, deleting, adding, or substituting an amino acid that restores a functional activity of the ABCC5 polypeptide having the amino acid sequence set forth in SEQ ID NO:2 or of the ABCG4 polypeptide having the amino acid sequence set forth in SEQ ID NO:4. Computer assisted three-dimensional molecular modeling may be employed to identify the amino acids residues to be changed that would increase the functional activity of the ABC transporter. Alternatively, in another embodiment, mutations can be introduced randomly along all or part of an ABC transporter coding sequence, such as by saturation mutagenesis. The resultant mutants can be screened for ABCC5 or ABCG4 transporter functional activity to identify mutants that retain activity. Following mutagenesis of either SEQ ID NOS: 1 or 3 or both, the encoded protein(s) can be expressed recombinantly and the activity of the protein can be determined.

In another embodiment, a functional activity of an ABC transporter polypeptide is a direct activity, such as an association with a membrane-associated protein and/or the transport of an endogenous or exogenous substrate across a biological membrane. In another embodiment, the ABC transporter polypeptide activity is the ability of the polypeptide to allosterically modify the function of other membrane proteins. For example, in some cells, modulation of inwardly-rectifying potassium channels by the ABC transporter ABCC8 (SURI) is an important biological mechanism for regulating insulin secretion from pancreatic β-cells (Babenko et al., Annu. Rev. Physiol. 60:667-87 (1998)). Thus, in this model, ABCC8 and other ABC transporters have multiple functions, one of which is to allosterically modify the function of the other membrane proteins. In certain preferred embodiments of the present invention, a direct functional activity of an ABCC5 transporter polypeptide or an ABCG4 polypeptide is modulated.

In another preferred embodiment, the functional activity of an ABC transporter is increased to alter the intracellular level of a catecholamine or a conjugate thereof, or to reduce catechoaminergic cell toxicity. Examples of drugs that increase ABC transporter activity are known in the art and include diazoxide, minoxidil, pinacidil, cromakalim (Babenko et al., J Biol. Chem. 275:717-20 (2000)).

In a preferred embodiment of the invention, the functional activity of an ABC transporter that is modulated to alter the intracellular level of a catecholamine or a conjugate thereof, or to reduce catecholaminergic cell toxicity, comprises modulating, preferably increasing, transport or translocation of a substrate across a cell membrane. A cell membrane includes the plasma membrane and membranes of subcellular organelles, for example, membranes of mitochondria, peroxisomes, and the endoplasmic reticulum. In certain other embodiments, the functional activity of an ABC transporter that is modulated comprises transport of a substrate out of a cell. In eukaryotes, most ABC transporter polypeptides transport or translocate (extrude, expel, sequester molecules from) compounds or molecules from the cytoplasm to the outside of the cell or into an extracellular compartment (e.g., the endoplasmic reticulum, mitochondria, peroxisomes). Not wishing to be bound by a particular mechanism, full transporters are generally found in the plasma membrane and transport molecules out of the cell and half transporters may be found in the membranes of subcellular organelles. Full transporters include, for example, twelve of the ABCA transporters, ABCB1, ABCB4, and the nine ABCC transporter polypeptides, to name a few. Half transporters include, for example, four ABC transporters in the ABCD subfamily, ABCB2, ABCB3, ABC7, ABC8, ABCG1, ABCG2, and ABCG4, to name a few.

A substrate of an ABC transporter may include a peptide or polypeptide, a hydrophobic compound (for example, a chemotherapy drug, e.g., colchicine, VP16, adriamycin, vinblastin, and lipids, steroids, xenobiotics, and hydrophobic peptides), ions (e.g., iron, zinc, chloride), anthracycline anti-cancer drugs, cholesterol, phospholipids, bile components, retinal and rhodopsin and conjugates thereof, sulfonylurea, long chain fatty acids, nucleosides, organic anions, 3,4-dihydroxyphenylacetic acid and homovanillic acid, and toxins. In a preferred embodiment of the invention, the substrate that is transported or translocated across a cellular membrane is a catecholamine or a conjugate thereof In certain preferred embodiments, the substrate is dopamine or a dopamine conjugate, such as glutathione conjugated dopamine-quinones or dopamine-semiquinones. In certain other preferred embodiments, the substrate is transported or extruded out of the cell.

In certain embodiments of the invention, the level of expression of an ABC transporter is modulated by altering degradation of wild-type or variant forms of an ABC transporter. In general, protein degradation occurs in lysozomes or proteosomes and may involve any number of different pathways, which may be dependent, in part, on the cell type and the location of the protein within the cell. In preferred embodiments, degradation of ABCC5 or ABCG4 transporter polypeptides is altered, more specifically, decreased (i.e., decreased in a statistically significant way). Degradation may be altered at any event in the catabolic pathway of ABCC5 or ABCG4. Examples include but are not limited to, alterations in (1) the folding of the native or variant protein; (2) targeting of the ABC transporter polypeptide for degradation; (3) proteolytic cleavage of the ABC transporter polypeptide; or (4) translocation of these ABC transporter polypeptides to their degradation site. Altering folding of native ABCC5 or ABCG4 may be performed by iterations of protein modeling and mutant construction to identify folding intermediates that may be affected to increase stability and activity of the ABC transporter polypeptide. Degradation of ABCC5 or ABCG4 may be altered by decreasing the rate of degradation by reducing the susceptibility of the ABC transporter polypeptide to thermal unfolding or by reducing its susceptibility to protease degradation, for example by decreasing the rate of deamidation of glutamine and asparagine residues. Degradation of a polypeptide may occur in a cell as a result of chemical aging or enzymatic modification of the polypeptide that labels it as a protein for degradation. Alternatively, attachment of certain membrane receptors for autophagy (process by which a discrete volume of cytoplasm is sequestered by the cytomembrane and which then fuses with a lysozome), or transient unfolding, or dissociation of a stabilizing ligand may lead to protein degradation. Any of these processes could be altered to decrease ABC polypeptide degradation. Degradation of polypeptides via lysozomes may be reduced by altering the pH of the lysozome. Alternatively, degradation may be altered by interfering with protein hydrolysis that occurs as part of an ATP-dependent proteolytic system. In further embodiments of the invention, an agent may modulate any event in the catabolic pathway of ABCC5 or ABCG4. An agent may constitutively modulate the degradation of an ABC transporter or may do so only under specific conditions or stresses. These agents or technologies may also be directed towards altering the degradation of any factor(s) that may directly or indirectly regulate the expression level and/or functional activity of these ABC transporters.

In other embodiments of the invention, the functional activity of an ABC transporter may be modulated, preferably increased, by removing, altering, or in some way abrogating, a suppressive effect of molecule or a cellular component or a condition, such as inflammation. By way of example, the pro-inflammatory cytokine interleukin (IL-) IL-6 mediates reduction in expression and activity of P-glycoprotein (ABCB1) (Sukahai et al., Mol. Cell Biol. Res. Commun. 4:248-56 (2000)).

In a preferred embodiment of the invention, catecholaminergic cell toxicity associated with the intracellular presence of a catecholamine, such as dopamine, or conjugates thereof is reduced according to a method comprising altering a functional activity of at least one ABC transporter polypeptide. A functional activity as defined herein may be altered according to the present invention. In another preferred embodiment, catecholaminergic cell toxicity associated with the intracellular presence of a catecholamine, such as dopamine, or conjugates thereof is reduced according to a method comprising altering, preferably increasing, the expression of an ABC transporter. Expression of an ABC transporter polypeptide, preferably ABCC5 and/or ABCG4 may be increased by transfecting or transforming the catecholaminergic cells or may be otherwise altered according to the present invention. In further preferred embodiments, also provided are methods to identify candidate agents that are capable of altering transcription or expression of an ABC transport gene, wherein altering (increasing or decreasing in a statistically significant manner, preferably increasing) transcription or expression alters, preferably decreases, catecholaminergic cell toxicity.

Catecholaminergic cell toxicity encompasses catecholaminergic cell death, such as neuronal cell death, which may be defined without limitation as an increased number of catecholaminergic cells dying, or an increased rate of cell death when compared to a normal cell. Catecholaminergic cell toxicity may also result from an accumulation of toxic molecules, such as radical oxygen species. Catecholaminergic cell toxicity may occur during oxidative stress, which may result in oxidative damage, such as depletion of glutathione; lipid, protein, and/or DNA oxidation; and deprivation of energy stores. In addition, catecholaminergic cell toxicity may be caused in part by an accumulation of catecholamines or conjugates thereof in the cell. In catecholaminergic cells, specifically neuronal cells, toxicity can result from an accumulation of dopamine or conjugates of dopamine, for example, dopamine-quinones and semiquinones. Catecholaminergic cell toxicity may be reduced by methods of the present invention to slow the rate of catecholaminergic cell death, to decrease the number of cells that die, to reduce the level of a catecholamine (preferably dopamine) or a conjugate thereof, to lessen the oxidative stress and damage to a cell, or any combination thereof, to lessen the toxicity and begin and continue to restore cell health and normal cellular functions.

The invention encompasses in vitro assays useful for assessing catecholaminergic cell toxicity, particularly dopamine-induced cytotoxicity. Cells (cell lines, primary or established cell cultures, immortalized cell lines, or primary cells) may be used to screen for agents (e.g., compounds, nucleotides, or antibodies) that alter the sensitivity of the cells to dopamine. Assays are used that employ various cell types (either cell lines or primary cells) from various host sources, such as any cells of prokaryotic or eukaryotic origin. For example, the cells contemplated for use in the present invention may be bacterial, such as E. coli; insect cells; yeast; or mammalian cells such as Chinese Hamster Ovary (CHO) or Human Embryonic Kidney 293 (HEK-292) cells. Preferably the cell is a mammalian cell, more preferably, a catecholaminergic cell of mammalian origin, and more preferably (but not limited to) a mammalian neuronal cell.

In a preferred embodiment of the invention, a method is provided for identifying an agent that is capable of altering catecholaminergic cell toxicity. The method comprises contacting a candidate agent and a first biological sample that comprises at least one cell under conditions and for a time sufficient to detect catecholaminergic cell toxicity in a cell from the first biological sample. The level of catecholaminergic cell toxicity in the first cell is compared to the level of catecholaminergic cell toxicity in one or more cells from a second biological sample that did not contact a candidate agent (negative control) and determining whether catecholaminergic cell toxicity was altered, preferable decreased, in a statistically meaningful way. In certain embodiments, catecholaminergic cell toxicity is altered by altering the expression of an ABC transporter polypeptide (preferably ABCC5 and/or ABCG4). Preferably, the expression of the ABC transporter polypeptide is increased. In certain other embodiments catecholaminergic cell toxicity is determined by measuring whether transport or translocation of a substrate of ABCC5 or an ABCG4 (or both) transporter polypeptide across a cell membrane is altered. A substrate is preferably a catecholamine or a conjugate thereof, and more preferably dopamine or a conjugate thereof. Transport or translocation may also encompass transport of the substrate out of the cell. In a preferred embodiment, an agent is identified that increases the capability of an ABC transporter to transport dopamine or dopamine conjugates out of a catecholaminergic cell. In additional embodiments, altering catecholaminergic cell toxicity comprises altering ATP hydrolysis activity of ABCC5 and/or ABCG4 transporter polypeptide. Alternatively, altering catecholaminergic cell toxicity comprises altering degradation (preferably decreasing in a statistically significant manner) of either the ABCC5 or the ABCG4 transporter polypeptide or both. The aforementioned methods and compositions relating to altering catecholaminergic cell toxicity are discussed in additional detail herein.

Assays may generally be performed using any of a variety of samples obtained from a biological source, such as eukaryotic cells, bacteria, viruses, extracts prepared from such organisms, and fluids found within living organisms. Biological samples that may be obtained from a patient include blood samples, biopsy specimens, tissue explants, organ cultures, and other tissue or cell preparations. A patient or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like. In certain preferred embodiments the patient or biological source is a human, and in certain preferred embodiments the biological source is a non-human animal that is a mammal, for example, a rodent (e.g., mouse, rat, hamster, etc.), an ungulate (e.g., bovine) or a non-human primate. In certain other preferred embodiments of the invention, a patient may be suspected of having or being at risk for having a disorder associated with catecholaminergic cell toxicity, or may be known to be free of a risk for or presence of such as disorder.

The particular cells employed for assessment of catecholaminergic cell toxicity, more specifically dopaminergic cell toxicity, may or may not have modified levels of one or more protein(s), such as ABC transporters, DAT proteins, or other proteins involved in dopaminergic pathways, as part of the creation of the cellular model. The cells can be created through transient or stable protein expression or, alternatively, can be isolated from a non-human transgenic organism. Other suitable cellular systems are known to those skilled in the art. In this in vitro cellular system, an agent that modulates dopaminergic cell toxicity may or may not modulate a function of an ABCC5 or an ABCG4 transporter polypeptide. The measurement of dopamine cytotoxicity in this cellular system is achieved by the use of various tools and techniques known to those skilled in the art such as immunological, enzymatic, and fluorescent techniques. Any standard technique known in the art for monitoring cellular susceptibility to a cytotoxin, or for monitoring accumulation of a cytotoxin within a cell, or for measuring the sequestration or extrusion of the cytotoxin can be adapted with routine experimentation to characterize catecholaminergic toxicity. In addition, methods known in the art for assessing cell viability may be performed as disclosed herein, such as monitoring viability using a colorimetric assay that measures conversion of methylthiazoletetrazolium (MTT) or a related substrate to a water-insoluble colored formazan derivative.

Candidate agents for use in these and related methods for identifying agents that modulate a functional activity of an ABC transporter, preferably, an ABCC5 or an ABCG4 transporter, or modulate expression of an ABC transporter, according to the present invention may be provided as “libraries” or collections of compounds, compositions, or molecules. The candidate agents of the present invention can be obtained using any of the numerous combinatorial library methods known in the art, including the following: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer, or small molecule libraries of compounds (Lam, K. S. Anticancer Drug Des. 12:145 (1997)). Examples of methods for the synthesis of molecular libraries can be found in the art, for example in DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and in Gallop et al., J. Med. Chem. 37:1233 (1994).

Libraries of compounds may be presented in solution (e.g., Houghten, Biotechniques 13:412-21 (1992)); on beads (Lam Nature 354:82-84 (1991)); on chips (Fodor, Nature 364:555-56 (1993)); on bacteria (Ladner, U.S. Pat. No. 5,223,409); spores (Ladner, supra); plasmids (Cull et al., Proc Natl. Acad Sci USA 89:1865-69 (1992)); or on phage (Scott and Smith, Science 249:386-90 (1990)); (Devlin, Science 249:404-06 (1990)); (Cwirla et al., Proc. Natl. Acad. Sci. 87:6378-82 (1990)); (Felici, J. Mol. Biol. 222:301-10 (1991)); (Ladner, supra.).

Candidate agents typically include compounds known in the art as “small molecules.” Such molecules have molecular weights less than 10⁵ daltons, preferably less than 10⁴ daltons, and still more preferably less than 10³ daltons.

Members of such libraries of test compounds can be administered to a plurality of samples, for use in the assays provided herein to determine their ability to enhance or inhibit transcription or expression (e.g., translation) of a reporter gene. Compounds so identified as capable of influencing ABCC5 or ABCG4 transporter expression levels are valuable for therapeutic and/or diagnostic purposes because such compounds may be used for treatment and/or detection of catecholaminergic cell toxicity-associated disorders. Such compounds are also valuable in research that is directed to further increasing a functional activity or the expression of an ABC transporter, and to refinements in the discovery and development of future compounds that exhibit greater specificity.

The process by which a polypeptide is expressed in a cell, that is, transcription of a gene (which includes regulatory sequences) into mRNA followed by translation of mRNA into protein, is known to be regulated. Regulation of gene transcription is generally coordinated by the actions of transcription factors, which are proteins that bind to and regulate a region of the gene known as the promoter. Transcriptional activity of the ABC transporter gene and regulation of its expression can be analyzed by methods known in the art. For example, primary transcripts may be analyzed by transcriptional run-on, which measures the intensity of transcription of a target gene under different experimental conditions (see Sambrook et al., Molecular Cloning: A Laboratory Manual (3^{rd} ed. 2001)). Typically, nuclei from cells expressing the ABC transporter gene are isolated and then incubated with a radiolabeled nucleotide, such as [³²]UTP. The transcriptional activity of the target gene is measured by hybridizing the radiolabeled RNAs to an excess of the target gene nucleic acid immobilized on a surface, for example, nitrocellulose or nylon membrane. The fraction of radioactivity that hybridizes to the immobilized DNA reflects the contribution of the target gene to the total transcriptional activity of the cell. Regulation of the gene is consequently detected by an increase or decrease in hybridization.

Those of skill in the art will readily appreciate that one can develop reporter assays that provide a measure of the regulation of expression of a gene, for example an ABC transporter gene (preferably, ABCC5 or ABCG4). As used herein, each of the terms “ABCC5” and “ABCG4 gene” refers to a nucleic acid molecule which includes an open reading frame encoding an ABCC5 or ABCG4 transporter protein, respectively, preferably a mammalian ABCC5 or ABCG4 transporter polypeptide, and can further include non-coding regulatory sequences, and introns. In one embodiment of the invention, reporter assays can be used to identify compounds that are capable of modifying the amount of ABC transporter mRNA, and ultimately ABC transporter protein in a cell. For example, a recombinant polynucleotide construct can be produced that comprises the promoter of a human ABC transporter polypeptide, the polynucleotide encoding the ABC transporter polypeptide, and a reporter gene wherein the translated protein produces a detectable signal in a cell.

Reporter genes or markers may be used to identify regulatory elements of a gene and to study promoters and enhancers and their interactions with cis-acting elements and trans-acting proteins, as well as measuring the effect of the addition of candidate agents (see Sambrook et al., supra)). A regulatory sequence of interest is joined (operably linked) to a reporter gene sequence present in an expression vector by standard recombinant methods. The resulting reporter gene recombinant is then introduced into an appropriate host cell line, and its expression is detected by measuring the reporter mRNA or the reporter protein, or in the instance when an enzyme reporter is used, by assaying for the relevant catalytic activity. The effect of the regulatory element on transcription is determined by measuring the detection signal or output that is distinguishable from background expression of proteins in the host cell. Appropriate controls for transcription assays are known in the art and may be incorporated according to the reporter system used. Suitable reporter genes may include genes encoding luciferase, beta-galactosidase, chloramphenicol acetyl transferase, secreted alkaline phosphatase, or in a non-enzymatic reporter system, green fluorescent protein (GFP) gene. Suitable expression systems are well known and may be prepared using well known techniques or obtained commercially (see Sambrook et al., supra).

The recombinant polynucleotide reporter construct may be inserted into cells (preferably of neuronal or neuroblastoma origin) that are then exposed to candidate agents (organic compounds, small molecules, peptides, genes, or others as described herein) in varying concentrations for varying periods of time. Using quantitative methods that are well known in the art, the ability of these compounds to alter expression of genes coupled to the promoter can be determined.

Other assays may be used to measure the ability of candidate agents to alter translation of mRNA into protein. For example, a person having skill in the art can measure the levels of specific mRNA species in cells using techniques such as Northern blot, quantitative PCR, ribonuclease protection, primer extension, or other techniques, and compare these with the levels of the protein (measured by immunoblot, ELISA, immunoprecipitation, immunochemistry, or other techniques known in the art) that is encoded by the particular mRNA species. If the candidate agent to which the cell is exposed alters translation of mRNA into protein, one would predict that the ratio of mRNA to protein would change when compared with a cell that was not exposed to the candidate agent. Other techniques, such as those disclosed in U.S. Pat. No. 6,107,029, “Universal method for detecting interactions between RNA molecules and RNA binding proteins,” incorporated herein by reference, can also be used to measure the ability of compounds to alter translation of mRNA.

According to particular embodiments of the invention, in vitro vesicular assays, such as those known to persons skilled in the art of ABC transporters, are used to screen for candidate agents that modulate a functional activity of ABCC5 and ABCG4. Assays are used that employ isolated cellular vesicles from various host sources such as any prokaryotic or eukaryotic cell. For example, these cells are bacterial cells, such as E. coli; insect cells; yeast; or other mammalian cells such as CHO or HEK-293 cells. Other suitable host cells are known to those skilled in the art. These host cell systems are used to prepare vesicles that contain either endogenous or over-expressed (transiently or stably) ABCC5 or ABCG4 transporter polypeptides. The methods of isolating cellular vesicles that may contain an ABC transporter are known to those skilled in the art; an example is provided in Germann et al., Biochemistry 29:2295-2303 (1990), which describes use of MDR1 overexpression in Sf9 insect cells. The measurement of ABCC5 or ABCG4 functional activity can be achieved in a variety of ways such as, but not limited to, detection of ATPase activity or assessment of direct substrate transport/translocation.

Agents, such as compounds or antibodies, that modulate the structure and/or activity of ABCC5 and ABCG4 in a manner that either prevents or reverses a propensity for these ABC transporters to aggregate are useful in practicing the invention. For example, results from one study suggest that ABCA4 will aggregate under conditions of photooxidation in the presence of all-trans-retinal (Sun and Nathans, J. Biol. Chem. 276:11766-74 (2001)). Similarly, ABCC5 or ABCG4 may aggregate under conditions of similar stresses that could lead to the development of agents that either directly or indirectly prevent or reverse this aggregation event.

The invention also provides diagnostic tools and methods for examining susceptibility to various catecholaminergic disease states associated with altered forms of ABCC5 or ABCG4. These alterations include, but are not limited to, genetic DNA polymorphisms or transcriptional, translational, or post-translational modifications in any of these ABC transporters. The diagnostic tools and methods include, but are not limited to, DNA or RNA sequence analysis, determination of splice-variants at the RNA or protein level or alternatively, protein analysis employed by standard antibody detection methods.

Also contemplated by the present invention are peptides, polypeptides, and other non-peptide molecules that specifically bind to an ABC transporter polypeptide. As used herein, a molecule is said to “specifically bind” to an ABCC5 transporter polypeptide, or an ABCG4 transporter polypeptide, if it reacts at a detectable level with ABCC5 or ABCG4, respectively, but does not react detectably with peptides containing an unrelated sequence, or a sequence of a different transporter. Preferred binding molecules include antibodies, which may be, for example, polyclonal, monoclonal, single chain, chimeric, anti-idiotypic, or CDR-grafted immunoglobulins, or fragments thereof, such as proteolytically generated or recombinantly produced immunoglobulin F(ab′)₂, Fab, Fv, and Fd fragments. Binding properties of an antibody to ABCC5 or to ABCG4 may generally be assessed using immunodetection methods including, for example, an enzyme-linked immunosorbent assay (ELISA), immunoprecipitation, immunoblotting and the like, which may be readily performed by those having ordinary skill in the art.

Methods well known in the art may be used to generate antibodies, polyclonal antisera, or monoclonal antibodies that are specific for an ABCC5 polypeptide or for an ABCG4 polypeptide. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second, distinct mammalian species. Most commonly, a chimeric antibody has murine variable region sequences and human constant region sequences. Such a murine/human chimeric immunoglobulin may be “humanized” by grafting the complementarity determining regions (CDRs) derived from a murine antibody, which confer binding specificity for an antigen, into human-derived V region framework regions and human-derived constant regions. Fragments of these molecules may be generated by proteolytic digestion, or optionally, by proteolytic digestion followed by mild reduction of disulfide bonds and alkylation. Alternatively, such fragments may also be generated by recombinant genetic engineering techniques.

As used herein, an antibody is said to be “immunospecific” or to “specifically bind” an ABC transporter polypeptide if it reacts at a detectable level with the ABC transporter polypeptide, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of binding partners or antibodies can be readily determined using conventional techniques, for example, those described by Scatchard et al. (Ann. N.Y Acad. Sci. USA 51:660 (1949)) or by surface plasmon resonance (BIAcore, Biosensor, Piscataway, N.J.) (Wolff et al., Cancer Res. 53:2560-65 (1993).

Antibodies may generally be prepared by any of a variety of techniques known to those having ordinary skill in the art. See, e.g., Harlow et al., Antibodies: A Laboratory Manual, Cold Spring Harbor Laboratory (1988). In one such technique, an animal is immunized with an ABCC5 transporter polypeptide or an ABCG4 transporter polypeptide as an antigen to generate polyclonal antisera. Suitable animals include, for example, rabbits, sheep, goats, pigs, cattle, and may also include smaller mammalian species, such as mice, rats, and hamsters, or other species.

An immunogen may be comprised of cells expressing an ABCC5 transporter polypeptide or an ABCG4 transporter polypeptide, purified or partially purified ABCC5 transporter polypeptide or ABCG4 transporter polypeptide, or variants or fragments thereof, or ABCC5 or ABCG4 peptides. ABCC5 or ABCG4 peptides may be generated by proteolytic cleavage or may be chemically synthesized. For instance, nucleic acid sequences encoding an ABCC5 transporter polypeptide or an ABCG4 transporter polypeptide are provided herein, such that those skilled in the art may routinely prepare these polypeptides for use as immunogens. Peptides may be chemically synthesized by methods as described herein and known in the art. Alternatively, peptides may be generated by proteolytic cleavage of a an ABCC5 transporter polypeptide or an ABCG4 transporter polypeptide, and individual peptides isolated by methods known in the art such as polyacrylamide gel electrophoresis or any number of liquid chromatography or other separation methods. Peptides useful as immunogens typically may have an amino acid sequence of at least 4 or 5 consecutive amino acids from an ABCC5 or an ABCG4 amino acid sequence such as those described herein, and preferably have at least 6, 7, 8, 9, 10, 11, 12, 14, 15, 16, 18, 19 or 20 consecutive amino acids of an ABCC5 transporter polypeptide or an ABCG4 transporter polypeptide. Certain other preferred peptide immunogens may comprise 21-25, 26-30, 31-35, 36-40, 41-50 or more consecutive amino acids of a an ABCC5 transporter polypeptide or an ABCG4 transporter polypeptide sequence. Polypeptides or peptides useful for immunization may also be selected by analyzing the primary, secondary, and tertiary structure of an ABCC5 transporter polypeptide or an ABCG4 transporter polypeptide according to methods known to those skilled in the art, in order to determine amino acid sequences more likely to generate an antigenic response in a host animal. See, e.g., Novotny, Mol. Immunol. 28:201-207 (1991); Berzofsky, Science 229:932-40 (1985).

Preparation of the immunogen for injection into animals may include covalent coupling of the ABCC5 transporter polypeptide or the ABCG4 transporter polypeptide polypeptide (or variant or fragment thereof), to another immunogenic protein, for example, a carrier protein such as keyhole limpet hemocyanin (KLH) or bovine serum albumin (BSA). In addition, the ABCC5 or ABCG4 peptide, polypeptide, or ABCC5- or ABCG4-expressing cells to be used as immunogen may be emulsified in an adjuvant. See, e.g., Harlow et al., Antibodies: A Laboratory Manual, Cold Spring Harbor Laboratory (1988). In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera out of the collected blood, and analyzing the sera in an immunoassay, such as an ELISA or Ouchterlony diffusion assay, or the like, to determine the specific antibody titer. Polyclonal antibodies that bind specifically to the ABCC5 or ABCG4 polypeptide or peptide may then be purified from such antisera, for example, by affinity chromatography using protein A, or the ABCC5 or ABCG4 polypeptide immobilized on a suitable solid support.

Monoclonal antibodies that specifically bind to ABCC5 or ABCG4 polypeptides or fragments or variants thereof, and hybridomas, which are immortal eukaryotic cell lines, that produce monoclonal antibodies having the desired binding specificity, may also be prepared, for example, using the technique of Kohler and Milstein (Nature, 256:495-97 (1976) and Eur. J. Immunol. 6:511-519 (1975)) and improvements thereto. Hybridomas producing monoclonal antibodies with high affinity and specificity for an ABCC5 transporter polypeptide or an ABCG4 transporter polypeptide are preferred. Hybridomas that produce monoclonal antibodies that specifically bind to an ABCC5 or an ABCG4 polypeptide or variant or fragment thereof are therefore contemplated by the present invention.

Human monoclonal antibodies may be generated by any number of techniques with which those having ordinary skill in the art will be familiar. Such methods include but are not limited to, Epstein Barr Virus (EBV) transformation of human peripheral blood cells (e.g., containing B lymphocytes) (see, e.g., U.S. Pat. No. 4,464,456); in vitro immunization of human B cells (Boemer et al., J. Immunol. 147:86-95 (1991)); fusion of spleen cells from immunized transgenic mice carrying human immunoglobulin genes inserted by yeast artificial chromosomes (YAC) (U.S. Pat. No. No. 5,877,397; Bruggemann et al., Curr. Opin. Biotechnol. 8:455-58 (1997); Jakobovits et al., Ann. N. Y Acad. Sci. 764:525-35 (1995)); isolation from human immunoglobulin V region phage libraries (U.S. Pat. No. 5,223,409; Huse et al., Science 246:1275-81 (1989); Kang et al., Proc. Natl. Acad. Sci. USA 88:4363-66 (1991); Hoogenboom et al., J. Molec. Biol. 227:381-88 (1992); Schlebusch et al., Hybridoma 16:47-52 (1997) and references cited therein), or other procedures as known in the art and based on the disclosure herein.

Chimeric antibodies, specific for an ABCC5 transporter polypeptide or an ABCG4 transporter polypeptide, including humanized antibodies, may also be generated according to the present invention. A chimeric antibody has at least one constant region domain derived from a first mammalian species and at least one variable region domain derived from a second, distinct mammalian species. See, e.g., Morrison et al., Proc. Natl. Acad. Sci. USA, 81:6851-55 (1984). In preferred embodiments, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region. See, e.g., Shin et al., Methods Enzymol. 178:459-76 (1989); Walls et al., Nucleic Acids Res. 21:2921-29 (1993). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for an ABCC5 transporter polypeptide or an ABCG4 transporter polypeptide when compared, for example, with either a non-human monoclonal antibody from which an ABCC5 transporter polypeptide or an ABCG4 transporter polypeptide binding variable region is obtained, or a chimeric antibody having such a V region and at least one human C region, as described above. See, e.g., Jones et al., Nature 321:522-25 1(986); Riechmann et al., Nature 332:323-27 (1988); Padlan et al., FASEB 9:133-39 (1995); Chothia et al., Nature, 342:377-383 (1989); Bajorath et al., Ther. Immunol. 2:95-103 (1995); EP-0578515-A3.

Within certain embodiments, the use of antigen-binding fragments of antibodies may be preferred. Such fragments include Fab fragments or F(ab′)₂ fragments, which may be prepared by proteolytic digestion with papain or pepsin, respectively. The antigen binding fragments may be separated from the Fc fragments by affinity chromatography, for example, using immobilized protein A or protein G, or immobilized ABCC5 or ABCG4 polypeptide, or a suitable variant or fragment thereof. Those having ordinary skill in the art can routinely and without undue experimentation determine what is a suitable variant or fragment based on characterization of affinity purified antibodies obtained, for example, using immunodetection methods as provided herein. An alternative method to generate Fab fragments includes mild reduction of F(ab′)₂ fragments followed by alkylation. See, e.g., Weir, Handbook of Experimental Immunology, Blackwell Scientific, Boston (1986).

According to certain embodiments, non-human, human, or humanized heavy chain and light chain variable regions of any of the above-described Ig molecules may be constructed as single chain Fv (sFv) polypeptide fragments (single chain antibodies). See, e.g., Bird et al., Science 242:423-26 (1988); Huston et al., Proc. Natl. Acad. Sci. USA 85:5879-83 (1988).

An ABCC5- or an ABCG4-binding immunoglobulin (or fragment thereof) as described herein may contain a detectable moiety or label such as an enzyme, cytotoxic agent, or other reporter molecule, including a dye, radionuclide, luminescent group, fluorescent group, or biotin, or the like. The ABCC5- or the ABCG4-specific immunoglobulin or fragment thereof may be radiolabeled for diagnostic or therapeutic applications. Techniques for radiolabeling of antibodies are known in the art. See, e.g., Adams, In Vivo 12:11-21 (1998); Hiltunen, Acta Oncol. 32:831-39 (1993). Therapeutic applications are described in greater detail below and may include use of the ABCC5- or the ABCG4-binding antibody (or fragment thereof) in conjunction with other therapeutic agents. The antibody or fragment may also be conjugated to a cytotoxic agent as known in the art and provided herein, for example, a toxin, such as a ribosome-inactivating protein, a chemotherapeutic agent, an anti-mitotic agent, an antibiotic or the like.

The invention also contemplates the generation of anti-idiotype antibodies that recognize an antibody (or antigen-binding fragment thereof) that specifically binds to an ABCC5 transporter polypeptide or an ABCG5 transporter polypeptide as provided herein, or a variant or fragment thereof. Anti-idiotype antibodies may be generated as polyclonal antibodies or as monoclonal antibodies by the methods described herein, using an anti-ABCC5 or anti-ABCG4 antibody (or antigen-binding fragment thereof) as immunogen. Anti-idiotype antibodies or fragments thereof may also be generated by any of the recombinant genetic engineering methods described above, or by phage display selection. An anti-idiotype antibody may react with the antigen binding site of the anti-ABCC5 or anti-ABCG4 antibody such that binding of the anti-ABCC5 or anti-ABCG4 antibody to a ABCC5 or ABCG4 polypeptide is competitively inhibited. Alternatively, an anti-idiotype antibody as provided herein may not competitively inhibit binding of an anti-ABCC5 or anti-ABCG4 antibody to an ABCC5 polypeptide or to an ABCG4 polypeptide, respectively.

As provided herein and according to methodologies well known in the art, polyclonal and monoclonal antibodies may be used for the affinity isolation of ABCC5 or ABCG4 polypeptides. See, e.g., Hermanson et al., Immobilized Affinity Ligand Techniques, Academic Press, Inc. New York (1992). Briefly, an antibody (or antigen-binding fragment thereof) may be immobilized on a solid support material, which is then contacted with a sample comprising the polypeptide of interest (e.g., an ABCC5 or ABCG4 polypeptide). Following separation from the remainder of the sample, the polypeptide is then released from the immobilized antibody.

Agents that modulate ABCC5 or ABCG4 expression or functional activity or both to treat a disorder that is associated with catecholaminergic cell toxicity resulting in impairment or degeneration of catecholaminergic neuronal or non-neuronal cells (i.e., adrenal cells) are also useful in practicing the invention. Such diseases include Parkinson's disease, Lewy Body dementia with or without Alzheimer's disease, spinocerebellar ataxia and other ataxias, dystonias, Brunner syndrome, and diseases of the adrenal gland. These agents are also suitable for treating diseases in which dysregulation or alteration of dopamine neuronal pathways has been suggested to be involved in disease pathology. For example, such disorders include but are not limited to schizophrenia, Tourette's syndrome, attention deficit disorder, alcoholism, drug addiction, Kelley-Seegmiller syndrome, and Lesch-Nyhan syndrome. In one embodiment of the invention, administering such an agent alleviates symptoms of a disorder. Alleviation of a symptom may encompass abrogation, lessening, or reduction in frequency of a particular symptom, and may also result in improvement in the quality of life. For example, symptoms of Parkinson's disease include shuffling gate, stooped posture, resting tremor, speech impediments, movement difficulties, and eventual slowing of mental processes, and dementia.

Non-human transgenic species that either overexpress or possess genetic knockout of ABCC5 or ABCG4 are also useful in practicing the invention. For example, the creation or use of a transgenic mouse that overexpresses either an ABCC5 transporter polypeptide or an ABCG4 transporter polypeptide is contemplated. Similarly, the creation or use of knockout mice that lack these endogenous ABC transporter genes is also contemplated. The animal is not limited to mice and also includes other non-human species such as transgenic or knockout nematodes (C. elegans), insects (Drosophila), or other animals (e.g., rats, guinea pigs, non-human primates). Specifically, ABCC5 or ABCG4 knockout mice provide inventive animal models for Parkinson's and other diseases affecting catecholaminergic neuronal or non-neuronal cells. Transgenic progeny or cells isolated from such animals are useful for studying the functional activities of an ABC transporter or expression of the transporter. Such animals and cells also may be used to identify and characterize candidate agents that modulate a functional activity or modulate expression, preferably increase a functional activity and/or expression, of an ABCC5 transporter polypeptide or an ABCG4 transporter polypeptide.

The invention provides a host cell that is a fertilized oocyte or an embryonic stem cell into which ABCC5- or ABCG4-coding sequences have been introduced. Such host cells can then be used to create non-human transgenic animals in which exogenous ABCC5 or ABCG4 transporter sequences have been introduced into their genome, or to create homologous recombinant animals in which endogenous ABCC5 or ABCG4 transporter sequences have been altered. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous ABCC5 or ABCG4 transporter gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an ABCC5- or an ABCG4-encoding nucleic acid into the male pronuclei of a fertilized oocyte, e.g., by microinjection, retroviral infection, and then allowing the oocyte to develop in a pseudopregnant female foster animal. The ABCC5 or ABCG4 transporter cDNA sequence of SEQ ID NOS:1 and 3, respectively, can be introduced as a transgene into the genome of a non-human animal. Alternatively, a nonhuman homologue of a human ABCC5 or ABCG4 transporter gene, such as a mouse or rat ABCC5 or ABCG4 transporter gene, can be used as a transgene. Alternatively, an ABCC5 or ABCG4 transporter gene homologue, such as another ABC transporter family member, can be isolated based on hybridization to the ABCC5 or ABCG4 transporter cDNA sequences of SEQ ID NOS:1 and 3, respectively, and used as a transgene. Intronic sequences and polyadenylation signals can also be included in the transgene to increase the efficiency of expression of the transgene. A tissue-specific regulatory sequence(s) can be operably linked to an ABCC5 or an ABCG4 transporter transgene to direct expression of an ABCC5 or an ABCG4 transporter polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al,. and in Hogan, B., Manipulating the Mouse Embryo, (Cold Spring Harbor Laboratory Press, Cold Spring Harbor, N.Y. (1986)). Similar methods are used for production of other transgenic animals. A transgenic founder animal can be identified based upon the presence of an ABCC5 or an ABCG4 transporter transgene in its genome and/or expression of ABCC5 or ABCG4 transporter mRNA in tissues or cells of the animals.

A transgenic founder animal can then be used to breed additional animals carrying the transgene. Also provided by the invention are transgenic animals carrying a transgene encoding an ABCC5 or ABCG4 transporter polypeptide that can further be bred to other transgenic animals carrying other transgenes. Interbreeding of ABCC5 or ABCG4 transgenic species with known animal models of Parkinson's or other diseases linked to alterations in catecholaminergic cells is also contemplated. For example, an ABCC5 or ABCG4 overexpressing transgenic animal can be interbred with an animal model of Parkinson's disease to determine if the Parkinsonian phenotype is ameliorated. Specifically, ABCC5 or ABCG4 transgenic or knockout mice are crossed with wild-type or mutant α-synuclein transgenic mice to create heterozygotic animals that may then show either an attenuated or potentiated parkinsonian phenotype, respectively. These procedures also include the creation of transgenic or knockout non-human species that have been designed to alter ABCC5 or ABCG4 activity either directly or indirectly through alteration in known molecules that interact with ABC transporters in general.

To create a homologous recombinant animal, a vector is prepared that contains at least a portion of an ABCC5 or an ABCG4 transporter gene into which a deletion, addition, or substitution has been introduced to thereby alter, e.g., functionally disrupt, the ABCC5 or ABCG4 transporter gene. The ABCC5 or ABCG4 transporter gene can be a human gene (e.g., the cDNA of SEQ ID NOS:1 or 3, respectively), but more preferably, is a non-human homologue of a human ABCC5 or ABCG4 transporter gene (e.g., a cDNA isolated by stringent hybridization with the nucleotide sequence of SEQ ID NOS:1 or 3, respectively). For example, a mouse ABCC5 or ABCG4 transporter gene can be used to construct a homologous recombination nucleic acid molecule, e.g., a vector, suitable for altering an endogenous ABCC5 or ABCG4 transporter gene in the mouse genome. In a preferred embodiment, the homologous recombination nucleic acid molecule is designed such that, upon homologous recombination, the endogenous ABCC5 or ABCG4 transporter gene is functionally disrupted (i.e., no longer encodes a functional protein; also referred to as a “knock out” vector). Alternatively, the homologous recombination nucleic acid molecule can be designed such that, upon homologous recombination, the endogenous ABCC5 or ABCG4 transporter gene is mutated or otherwise altered but still encodes functional protein (e.g., the upstream regulatory region can be altered to thereby alter the expression of the endogenous ABCC5 or ABCG4 transporter protein). In the homologous recombination nucleic acid molecule, the altered portion of the ABCC5 or ABCG4 transporter gene is flanked at its 5′ and 3′ ends by additional nucleic acid sequence of the ABCC5 or ABCG4 transporter gene, respectively, to allow for homologous recombination to occur between the exogenous ABCC5 or ABCG4 transporter gene carried by the homologous recombination nucleic acid molecule and an endogenous ABCC5 or ABCG4 transporter gene in a cell, e.g., an embryonic stem cell. The additional flanking ABCC5 or ABCG4 transporter nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas et al., Cell 51:503 (1987) for a description of homologous recombination vectors). The homologous recombination nucleic acid molecule is introduced into a cell, such as an embryonic stem cell line (e.g., by electroporation) and cells in which the introduced ABCC5 or ABCG4 transporter gene has homologously recombined with the endogenous ABCC5 or ABCG4 transporter gene are selected (see e.g., Li, E. et al., Cell 69:915 (1992)). The selected cells can then injected into a blastocyst of an animal (e.g., a mouse) to form aggregation chimeras (see, e.g., Bradley, A. in Teratocareinomas and Embryonic Stem Cells: A Practical Approach, E. J. Robertson, ed., IRL, Oxford (1987)) pp. 113-52). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, such as vectors, or homologous recombinant animals are described further in Bradley, A., Current Opinion in Biotechnology 2:823-29 (1991) and in PCT International Publication Nos. WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, see, e.g., Lakso et al., Proc. Natl. Acad. Sci. USA 89:6232-36 (1992). Another example of a recombinase system is the FLP recombinase system of Saccharomyces cerevisiae (O'Gorman et al., Science 251:1351-55 (1991)). If a cre/loxP recombinase system is used to regulate expression of the transgene, animals containing transgenes encoding both the Cre recombinase and a selected protein are required. Such animals can be provided through the construction of “double” transgenic animals, such as by mating two transgenic animals, one containing a transgene encoding a selected protein and the other containing a transgene encoding a recombinase.

Clones of the non-human transgenic animals described herein can also be produced according to the methods described in Wilmut et al., Nature 385:810-13 (1997) and PCT International Publication Nos. WO 97/07668 and WO 97/07669. In brief, a cell, such as a somatic cell from the transgenic animal can be isolated and induced to exit the growth cycle and to enter G_{O} phase. The quiescent cell can then be fused, for example, through the use of electrical pulses, to an enucleated oocyte from an animal of the same species from which the quiescent cell is isolated. The reconstructed oocyte is cultured such that it develops to morula or blastocyte and is then transferred to pseudopregnant female foster animal. The offspring borne of this female foster animal will be a clone of the animal from which the cell, such as the somatic cell, is isolated.

In another approach, agents (compounds, nucleotides or antibodies, etc.) are employed to alter the expression and/or functional activity of ABCC5 or ABCG4 in order to mimic disease state phenotype and cellular alterations indicative of Parkinson's or other diseases affecting catecholaminergic neuronal and non-neuronal cells. Interbreeding of ABCC5 or ABCG4 transgenic species with known animal models of Parkinson's disease or other diseases associated with alterations in catecholaminergic neuronal or non-neuronal cells is also contemplated. Specifically, ABCC5 or ABCG4 transgenic mice are crossed with wild-type or mutant α-synuclein transgenic mice to create heterozygotic animals that may then show an attenuated α-synuclein mutant phenotype (parkinsonianism). These procedures also include the creation of transgenic or knockout non-human species that have been designed to alter ABCC5 or ABCG4 activity either directly or indirectly through alteration in known molecules that interact with ABC transporters in general.

In another embodiment of the invention, candidate agents (e.g., compounds, nucleotides, small molecules, or antibodies as described herein) that alter the expression and/or functional activity of an ABCC5 or an ABCG4 transporter polypeptide are used to either attenuate or recapitulate Parkinson's disease phenotype when administered to various transgenic or non-transgenic animal models. Expression or a functional activity or both of an ABCC5 or an ABCG4 transporter polypeptide may be altered by methods, including but not limited to, the focal infusion of neurotoxins. In an embodiment of the invention, agents can be tested in any animal model resulting from the interbreeding of wild-type or ABCC5 or ABCG4 transgenic animals with non-transgenic animals that represent non-transgenic animal models of Parkinson's disease (or any disorder having catecholaminergic cell pathology). Also contemplated by the present invention, are procedures that include testing of candidate agents in transgenic or non-transgenic non-human species that have been designed to alter ABCC5 or ABCG4 activity, either directly or indirectly, through alteration in molecules known to interact with ABC transporters in general.

Thus, the present invention provides a method for identifying an agent capable of altering (increasing or decreasing in a statistically significant manner, preferably decreasing) catecholaminergic cell toxicity in a cell of a non-human transgenic or non-transgenic animal that has a disorder associated with catecholaminergic cell toxicity, wherein the method comprises treating at least one, preferably a sufficient number of animals to provide statistically significant data, with a candidate agent; measuring the level of catecholaminergic cell toxicity in the animals; comparing the level of catecholaminergic cell toxicity to that observed in animals that have a disorder associated with catecholaminergic cell toxicity but which did not receive the candidate agent. A reduction in the level of catecholaminergic cell toxicity indicates that the agent alters toxicity. In a certain embodiment of the invention, the level of catecholaminergic cell toxicity is measured by a method comprising detecting extrusion of a catecholamine, or a conjugate thereof, from at least one neuronal cell of the animal(s) having a disorder associated with catecholaminergic cell toxicity and also receiving the candidate agent, and comparing the level of catecholamine or a conjugate thereof of animals from a second group that have the disorder but which did not receive the candidate agent. In a preferred embodiment of the invention, the level of the catecholamine dopamine, or a conjugate of dopamine, is measured to determine catecholaminergic cell toxicity. Reduction of catecholaminergic cell toxicity also can be determined by additional methods known in the art and disclosed herein, for example, by quantifying the number of animals that die or the number of cells (preferably catecholaminergic cells, and more preferably neuronal cells) of the animal that die; by observing behaviors of the animals, for example, whether the animals feed normally, and/or whether the animals are active or lethargic; and other methods.

The present invention is also directed to gene therapy. For the purposes of the present invention, gene therapy refers to the transfer and stable insertion of new genetic information into cells for the therapeutic treatment of diseases or disorders. A foreign sequence or gene is transferred into a cell that may or may not proliferate to spread the new sequence or gene throughout the cell population. Sequences include a sense or antisense sequence of an ABC transporter (preferably, ABCC5 or ABCG4). Known methods of gene transfer include microinjection, electroporation, liposomes, chromosome transfer, transfection techniques, calcium-precipitation transfection techniques, and the like.

A disorder or disease such as Parkinson's disease characterized by loss of catecholaminergic neurons may result from a loss of gene function (as a result of a mutation for example), or a gain of gene function (as a result of an extra copy of a wild-type gene), or overexpression of a gene (as a result of a mutation in a promoter). Expression may be modulated (altered) by activating or deactivating regulatory elements, such as a promoter. A mutation or polymorphism may be corrected by replacing the mutated sequence with a wild-type sequence or inserting an antisense sequence to bind to an overexpressed sequence or to a regulatory sequence.

Numerous techniques are known in the art for the introduction of foreign genes into cells and may be used to construct the recombinant cells for the purposes of gene therapy, in accordance with this embodiment of the invention. Techniques that may be used include but are not limited to chromosome transfer (e.g., cell fusion, chromosome-mediated gene transfer, micro cell-mediated gene transfer), physical methods (e.g., transfection, spheroplast fusion, microinjection, electroporation, liposome carriers), viral vector transfer (e.g., recombinant DNA viruses, recombinant RNA viruses) and the like (reviewed in Dyer et al, Molecular Therapy: the Journal of the American Society of Gene Therapy. 1:213-24 (2000)), and may be particularly suitable for the treatment of Parkinson's disease (Bohn M. C., Molecular Therapy: the Journal of the American Society of Gene Therapy 1:494-96 (2000)). The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. Proc. Natl. Acad. Sci. USA 91:3054-57 (1994)). The pharmaceutical preparation of the gene therapy vector can include the gene therapy vector in an acceptable diluent, or can comprise a slow release matrix in which the gene delivery vehicle is imbedded. Alternatively, when the complete gene delivery vector can be produced intact from recombinant cells, such as retroviral vectors, the pharmaceutical preparation can include one or more cells that produce the gene delivery system.

The invention also contemplates treating a subject who has a disorder associated with catecholaminergic cell toxicity, such as Parkinson's disease, by providing the subject in need thereof, cells that express an ABC transporter polypeptide, such as ABCC5 or ABCG4. Such ex vivo procedures may be used in which cells are removed from a host, modified, and placed into the same or another host animal. Alternatively, a cell from a host may be selected for on the basis of an increased expression level of an ABCC5 or ABCG4 polypeptide, and then modified if necessary, and placed into the same or another host animal. Protocols for viral, physical, and chemical methods of uptake are well known in the art.

Such pharmaceutical compositions typically comprise the recombinant nucleic acid construct, a polypeptide, antibody or other agent, such as a small molecule, and a pharmaceutically acceptable carrier. As used herein the term “pharmaceutically acceptable carrier” is intended to include any and all solvents, dispersion media, coatings, antibacterial and antifingal agents, isotonic, and absorption delaying agents, and the like, compatible with pharmaceutical administration. The use of such media and agents for pharmaceutically active substances is well known in the art. Except insofar as any conventional medium or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, such as intravenous, intradermal, intrathecal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates, and agents for the adjustment of tonicity such as sodium chloride or dextrose. The pH can be adjusted with an acid or a bases, such as hydrochloric acid or sodium hydroxide, respectively. The parenteral preparation can be enclosed in ampoules, disposable syringes, or multiple dose vials made of glass or plastic.

Pharmaceutical compositions suitable for injectable use include sterile aqueous solutions (when the composition is water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. The pharmaceutical composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with an excipient and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser that contains a suitable propellant, e.g., a gas such as carbon dioxide, or a nebulizer. Alternatively, an external source of pressure is included such as a pump.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation (Mountain View, Calif.). Liposomal suspensions can also be used as pharmaceutically acceptable carriers. These can be prepared according to methods known to those skilled in the art, for example, as described in U.S. Pat. No. 4,522,811.

In certain embodiments, oral or parenteral compositions are formulated in dose unit form for ease of administration and uniformity of dose. Dose unit form as used herein refers to physically discrete units suited as unitary doses for the subject to be treated, wherein each unit contains a predetermined quantity of active compound calculated to produce the desired therapeutic effect in association with the required pharmaceutical carrier. The specification for the dose unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, such as for determining the LD50 (the dose lethal to 50% of the population) and the ED50 (the dose therapeutically effective in 50% of the population). The dose ratio between toxic and therapeutic effects is the therapeutic index, which can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, to reduce side effects.

The data obtained from the cell culture assays and animal studies can be used in formulating a range of doses for use in humans. The dose of such compounds lies preferably within a range of circulating concentrations that include the ED50 with little or no toxicity. The dose may vary within this range depending upon the dose form employed and the route of administration used. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions may be administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the agent(s) in an amount sufficient to provide therapeutic and/or prophylactic benefit (e.g., an improved clinical outcome, such as more frequent complete or partial remissions, or longer disease-free and/or overall survival). For prophylactic use, a dose should be sufficient to prevent, delay the onset of or diminish the severity of a disease or disorder associated with catecholaminergic cell toxicity.

The following Examples are offered by way of illustration and not by way of limitation.

### EXAMPLES

### Example 1

### Effect of ABC Transporter Expression on Dopamine Toxicity in Catecholaminergic Cells

This Example demonstrates the effect on dopamine toxicity in a neuroblastoma cell that is transfected with expression vectors encoding ABCC5 and ABCG4 transporter polypeptides.

The cell line used in Examples 1-3 is known as hDAT-SK-N-MC (hDAT) cells (Pifl et al., J. Neurosci. 13:4246-53 (1993); Silvia et al., Neuroscience 76:737-47 (1997)). Parental SK-N-MC cells (a human neuroblastoma line exhibiting catecholaminergic properties) were transfected with human dopamine transporter (hDAT) cDNA, resulting in a stable cell line expressing hDAT. The hDAT cell line was maintained in DMEM (Invitrogen Life Technologies, Gaithersburg, Md.) supplemented with sodium pyruvate (1 mM), glutamine (1 mM), 10% fetal bovine serum (FBS) and G418 (250 μg/ml). Cells were maintained in antibiotic-free media before being used in experiments and were seeded in phenol-free culture medium at a density of 8.5×10⁴ cells/well in 24-well tissue culture plates.

DA (Sigma-Aldrich, St. Louis, Mo.) stock solution (10 mM) was prepared in phenol-free and serum-free culture medium. The DAT inhibitor N-[l-(2-benzo(b)thiophenyl)cyclohexyl]piperidine (BTCP) maleate (Tocris Cookson, Ellisville, MO) (Vignon et al., Eur. J Pharmacol. 148:427-36 (1988)) was dissolved in ethanol to make a 10 mM stock solution.

Human ABC transporter cDNAs were subcloned into the plasmid expression vector pCEP4 or pcDNA3.1 (Invitrogen Life Technologies) according to instructions provided by the vendor and according to standard molecular biology techniques. All cloning, mutagenesis, and transfection methods were performed following standard methods known to those in the molecular biology art. Constructs were prepared containing a polynucleotide sequence (SEQ ID NO:1) encoding ABCC5 transporter polypeptide (SEQ ID NO:2), a polynucleotide sequence (SEQ ID NO:3) encoding ABCG4 transporter polypeptide (SEQ ID NO:4), and polynucleotide sequences encoding two other ABC transporters, ABCx and ABCy. Plasmid constructs used as controls included constructs encoding either no ABC transporter or a non-functional ABC transporter (ABCz) obtained by introducing mutations into the Walker A and Walker B motifs of its ATP binding site. hDAT cells seeded as described above were transiently transfected using the lipophilic reagent SuperFect (QIAGEN, Valencia, Calif.) and 0.75 μg of plasmid construct per well diluted in culture medium. After a 3 hour incubation (37° C.), the transfection solution was replaced by culture medium containing 15% FBS. Following a 48 hour recovery period to allow the expression of the human transgene, cultures were treated with dopamine (DA) (50 μM) for 24 hours in media containing 4% FBS. In one culture containing hDAT cells transfected with the empty vector, 50 nM BTCP (DAT inhibitor) was added one hour prior to the addition of DA.

DA-cytotoxicity was assessed using the colorimetric methylthiazoletetrazolium (MTT) assay. Briefly, media containing DA or DA plus BTCP was gently removed before 0.4 ml of 0.5 mg/ml MTT in serum- and phenol-free media was added to each well. Cells were incubated at 37° C. for 2 hours to allow the enzymatic conversion by cellular dehydrogenase activity in viable cells of the yellow tetrazolium salt MTT to purple formazan crystals. The resulting purple formazan crystals were solubilized by addition of 0.4 ml/well of lysis buffer (10% Triton X-100, 1 M HCl in isopropanol). Absorbance at 570 nm was quantified using a Victor² microplate reader (EG&G Wallac, now Perkin-Elmer Life Sciences, Boston, Mass.).

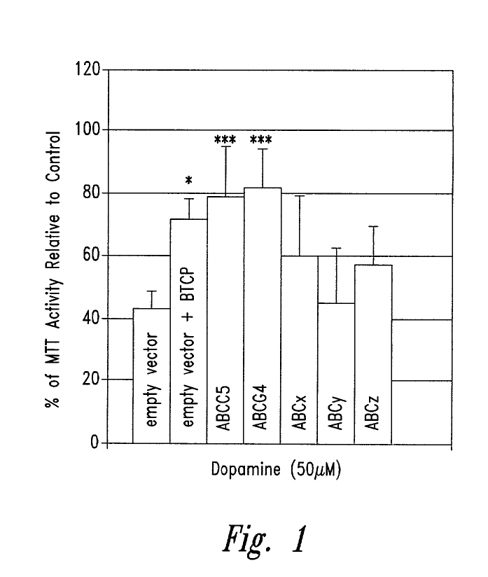

The percent of MTT conversion measured for each sample containing cells transfected with an ABC transporter was compared with the percent of MTT conversion measured in the empty vector control group. Data were combined from 1-3 experiments (n=4-14). Data were analyzed by one-way ANOVA with Tukey's post hoc analysis using GraphPad software (GraphPad Software, San Diego, Calif.). Results were considered significant at p<0.05. The effect of expression of the ABC transporter polypeptides on dopamine toxicity in hDAT cells is presented in FIG. 1.

### Example 2

### Effect of ABC Transporters on Dopamine Toxicity in Catecholaminergic Cells in the Presence of ABC Transporter Inhibitors

This Example demonstrates the effect on dopamine toxicity in a neuroblastoma cell that is transfected with expression vectors encoding ABCC5 and ABCG4 transporter polypeptides in the presence of ABC transporter inhibitors.

Stock solutions of two ABC transporter inhibitors, glipizide (200 mM, Tocris Cookson) and probenecid (10 mM, Sigma-Aldrich) were prepared in dimethylsulfoxide (DMSO) and phenol- and serum-free culture medium, respectively (Lamensdorf et al., Naunyn-Schmiedeberg's Arch. Pharmacol. 361:654-64 (2000); Jedlitschky et al., J. Biol. Chem. 275:30069-74 (2000)).

hDAT cells transiently transfected with the pCEP4 empty construct or expression vectors encoding the ABCC5 or ABCG4 ABC transporters, prepared as described in Example 1, were treated with DA (50 μM) for 24 hours in the presence or absence of ABC transporter inhibitors. Two hours prior to the addition of DA to the samples, glipizide (250 μM) or probenecid (500 μM) were added to transfected hDAT cells. DA-induced cytotoxicity was then assessed using the MTT assay as described in Example 1. FIG. 2 represents the effect of inhibiting the ABC transporter activity of ABCC5 and ABCG4 on dopamine-treated hDAT cells pre-treated with glipizide (FIG. 2A) or probenecid (FIG. 2B). The percent MTT activity for transfected hDAT cells is relative to the percent MTT activity measured for those transfected hDAT cells that received neither DA nor an ABC transporter inhibitor (“No treatment”). The data represent one experimental trial (n=2).

### Example 3

### Effect of ABC Transporter Mutants on Dopamine Toxicity in Catecholaminergic Cells

This Example describes the effect on dopamine toxicity in a neuroblastoma cell line when the cells are transfected with non-functional ABC transporter mutants.

An ABCG4 transporter mutant was prepared according to standard site-directed mutagenesis methods. The mutant ABCG4 transporter polypeptide (SEQ ID NO:8) contained two mutations, the lysine at position 108 in wild-type ABCG4 polypeptide (SEQ ID NO:4) was changed to arginine (K108R) and the aspartate residue at position 225 was changed to asparagine (D225N). Mutant ABCC5 transporter polypeptide (SEQ ID NO:6) was obtained by deleting 126 amino acid residues at the C-terminal portion (all residues after cysteine at position 1311 were deleted, i.e., wild-type amino acids 1312-1437 were deleted) of the wild-type ABCC5 polypeptide (SEQ ID NO:2), which contained the Walker A and Walker B regions. For subcloning purposes, a nucleotide sequence encoding 14 amino acids (IEAGKAESRHDKIH) (SEQ ID NO:9) was added following the cysteine residue at position 1311.

hDAT cells were transfected with recombinant expression constructs containing the mutant ABCC5 polynucleotide sequence (SEQ ID NO:5) and the mutant ABCG4 polynucleotide sequence (SEQ ID NO:7) according to the methods described in Example 1. Transfected cells were exposed to DA (50 μM) for 24 hours in culture media containing 4% serum (see procedures in Example 1). DA-induced cytotoxicity was then assessed by the MTT assay as described in Example 1.

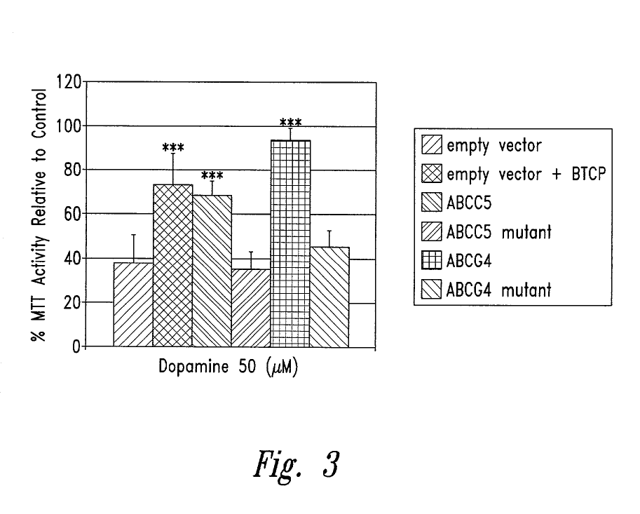

The percent of MTT conversion in the presence of DA that was measured for each sample containing cells transfected with an ABC transporter, ABC transporter mutant, or empty vector was compared to the percent of MTT conversion measured in the absence of DA. Data were analyzed as described in Example 1. The effect of expression of the wild-type and mutant ABC transporter polypeptides on dopamine toxicity in hDAT cells is presented in FIG. 3.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for modulating an intracellular level of a catecholamine, or a conjugate thereof, in a cell, comprising modulating a functional activity of at least one ABC transporter polypeptide of the cell, and thereby modulating the intracellular level of the catecholamine, or a conjugate thereof, in the cell.

2. A method for modulating an intracellular level of a catecholamine, or a conjugate thereof, in a cell, comprising modulating a level of expression of at least one ABC transporter polypeptide of the cell, and thereby modulating the intracellular level of the catecholamine, or a conjugate thereof, in the cell.

3. The method of either claim 1 or claim 2, wherein the ABC transporter polypeptide is selected from the group consisting of an ABCC5 transporter and an ABCG4 transporter.

4. The method of claim 2 wherein the level of expression of the ABC transporter polypeptide is increased.

5. The method of claim 4 wherein the level of expression of the ABC transporter polypeptide is increased by transfecting or transforming the cell with a recombinant nucleic acid construct comprising a polynucleotide encoding the ABC transporter polypeptide.

6. The method of claim 5 wherein the recombinant nucleic acid construct comprises a nucleotide sequence having at least 80% identity with a sequence as set forth in any one of the sequences selected from the group consisting of SEQ ID NO:1 and SEQ ID NO:3.

7. The method of claim 6 wherein the recombinant nucleic acid construct further comprises a promoter operably linked to the nucleotide sequence.

8. The method of either claim 1 or claim 2, wherein the cell is a mammalian cell.

9. The method of claim 8 wherein the mammalian cell is a catecholaminergic cell.

10. The method of claim 9 wherein the catecholaminergic cell is a neuronal cell.

11. The method of either claim 1 or claim 2, wherein the catecholamine is dopamine.

12. The method of claim 1 wherein the functional activity of at least one ABC transporter polypeptide is increased.

13. The method of claim 1 wherein the functional activity of the ABC transporter polypeptide comprises transport or translocation of a substrate across a cell membrane.

14. The method of claim 1 wherein the functional activity of the ABC transporter polypeptide comprises transport of a substrate out of the cell.

15. The method of either claim 13 or claim 14, wherein the substrate comprises a catecholamine, or a conjugate thereof.

16. The method of either claim 13 or claim 14, wherein the substrate comprises dopamine, or a conjugate thereof.

17. The method of claim 1 wherein the functional activity of the ABC transporter polypeptide comprises ATP hydrolysis.

18. The method of claim 2 wherein modulating a level of expression of the ABC transporter polypeptide comprises altering degradation of the ABC transporter polypeptide.

19. A method for reducing catecholaminergic cell toxicity associated with the presence of a catecholamine or a conjugate thereof in a cell, said method comprising modulating a functional activity of at least one ABC transporter polypeptide in the cell, and thereby reducing catecholaminergic cell toxicity.

20. A method for reducing catecholaminergic cell toxicity associated with the presence of a catecholamine or a conjugate thereof in a cell, said method comprising modulating a level of expression of at least one ABC transporter polypeptide in the cell, thereby reducing catecholaminergic cell toxicity.

21. The method of either claim 19 or claim 20 wherein the ABC transporter polypeptide is selected from the group consisting of an ABCC5 transporter and an ABCG4 transporter.

22. The method of claim 20 wherein the level of expression of the ABC transporter polypeptide is increased.

23. The method of claim 22 wherein the level of expression is increased by transfecting or transforming the cell with a recombinant nucleic acid construct comprising a polynucleotide encoding the ABC transporter polypeptide.

24. The method of claim 23 wherein the recombinant nucleic acid construct comprises a nucleotide sequence having at least 80% identity with a sequence as set forth in any one of the sequences selected from the group consisting of SEQ ID NO:1 and SEQ ID NO:3.

25. The method of claim 24 wherein the recombinant nucleic acid construct further comprises a promoter operably linked to the nucleotide sequence.

26. The method of either claim 19 or claim 20 wherein the catecholamine is dopamine.

27. The method of claim 19 wherein the functional activity of at least one ABC transporter polypeptide is increased.

28. The method of claim 19 wherein the functional activity of the ABC transporter polypeptide comprises transport or translocation of a substrate across a cell membrane.

29. The method of claim 19 wherein the functional activity of the ABC transporter polypeptide comprises transport of a substrate out of the cell.

30. The method of either claim 28 or claim 29, wherein the substrate comprises a catecholamine, or a conjugate thereof.

31. The method of either claim 28 or claim 29, wherein the substrate comprises dopamine, or a conjugate thereof.

32. The method of claim 19 wherein the functional activity of the ABC transporter polypeptide comprises ATP hydrolysis.

33. The method of claim 20, wherein modulating a level of expression of an ABC transporter polypeptide comprises altering degradation of the ABC transporter polypeptide.

34. A method for alleviating symptoms of a disorder associated with catecholaminergic cell toxicity in a subject, said method comprising administering to the subject in need thereof at least one agent that modulates a functional activity of an ABC transporter polypeptide.

35. A method for alleviating symptoms of a disorder associated with catecholaminergic cell toxicity in a subject, said method comprising administering to the subject in need thereof at least one agent that modulates a level of expression of an ABC transporter polypeptide.

36. The method of either claim 34 or claim 35 wherein the ABC transporter polypeptide is selected from the group consisting of an ABCC5 transporter and an ABCG4 transporter.

37. The method of either claim 34 or claim 35 wherein the disorder is selected from the group consisting of Parkinson's disease, Lewy Body dementia, spinocerebellar ataxia, Brunner syndrome, an adrenal gland disorder, schizophrenia, Tourette's syndrome, attention deficit disorder, alcoholism, drug addiction, Kelley-Seegmiller syndrome, and Lesch-Nyhan syndrome.

38. The method of claim 37 wherein the disorder is selected from the group consisting of Parkinson's disease, Lewy Body dementia, an adrenal gland disorder, schizophrenia, Tourette's syndrome, attention deficit disorder, alcoholism, and drug addiction.

39. The method of either claim 34 or claim 35 wherein the disorder is Parkinson's disease.

40. The method of claim 34 wherein the functional activity of the ABC transporter polypeptide is increased.

41. The method of claim 34 wherein the functional activity of the ABC transporter polypeptide comprises transport or translocation of a substrate across a cell membrane.

42. The method of claim 34 wherein the functional activity of the ABC transporter polypeptide comprises transport of a substrate out of the cell.

43. The method of either claim 41 or claim 42 wherein the substrate comprises a catecholamine or a conjugate thereof.

44. The method of either claim 41 or claim 42 wherein the substrate comprises dopamine, or a conjugate thereof.

45. The method of claim 34 wherein the functional activity of the ABC transporter polypeptide comprises ATP hydrolysis.

46. The method of claim 35 wherein the level of expression of the ABC transporter polypeptide is increased.

47. The method of claim 35 wherein modulating the level of expression of the ABC transporter polypeptide comprises altering degradation of the ABC transporter polypeptide.

48. A non-human transgenic animal for identifying an agent that modulates catecholaminergic cell toxicity in a catecholaminergic cell.

49. The non-human transgenic animal of claim 48 wherein the transgenic animal overexpresses an ABCG4 transporter polypeptide.

50. The non-human transgenic animal of claim 48, wherein the transgenic animal is a knockout animal lacking a gene encoding an ABCG4 transporter polypeptide.

51. A method for identifying an agent that is capable of modulating catecholaminergic cell toxicity in a cell of a non-human animal, comprising:
(a) treating at least one first non-human animal having a disorder associated with catecholaminergic cell toxicity with a candidate agent;
(b) measuring the level of catecholaminergic cell toxicity in at least one first catecholaminergic cell of the first animal; and
(c) comparing the level of catecholaminergic cell toxicity in the first animal with the level of catecholaminergic cell toxicity in at least one second catecholaminergic cell of at least one second non-human animal having a disorder associated with catecholaminergic cell toxicity, wherein the second animal was not treated with the candidate agent, and wherein a reduction in the level of catecholaminergic cell toxicity in the first catecholaminergic cell of the first animal compared with the level of catecholaminergic cell toxicity in the second catecholaminergic cell of the second animal indicates that the agent modulates catecholaminergic cell toxicity.

52. The method of claim 51 wherein the level of catecholaminergic cell toxicity is measured by a method comprising:
(a) detecting extrusion of a catecholamine, or a conjugate thereof, from at least one first neuronal cell of the first animal; and
(b) comparing a level of catecholamine, or a conjugate thereof, extruded from the first neuronal cell with a level of catecholamine, or a conjugate thereof, from at least one second neuronal cell of the second animal that was not treated with the candidate agent,
wherein an increased level of catecholamine, or a conjugate thereof, extruded from the first neuronal cell of the first animal treated with the agent compared with the level of catecholamine or a conjugate thereof extruded from the second neuronal cell of the second animal that was not treated with the candidate agent indicates that the agent modulates the toxicity of catecholamine or a conjugate thereof in the first neuronal cell.

53. The method of claim 52 wherein the catecholamine is dopamine.

54. The method of claim 51 wherein the non-human animal is the transgenic animal according to claim 48.

55. A method for identifying an agent that is capable of modulating expression of an ABCG4 transporter polypeptide, comprising:
(a) contacting a candidate agent and a first biological sample comprising at least one first cell that is capable of expressing the ABCG4 transporter polypeptide, under conditions and for a time sufficient to detect ABCG4 transporter expression; and
(b) comparing a level of ABCG4 transporter expression in the first cell with a level of ABCG4 transporter expression in at least one second cell in a control sample that has not been contacted with the candidate agent, wherein an increased level of ABCG4 transporter expression in the presence of the candidate agent relative to the level of ABCG4 transporter expression in the second cell in the control sample that has not been contacted with the candidate agent indicates that the agent is capable of modulating ABCG4 transporter expression.

56. A method for identifying an agent that is capable of modulating transcription or expression of an ABC transporter gene, wherein modulating transcription or expression of the ABC transporter gene modulates catecholaminergic cell toxicity in a cell, comprising:
(a) contacting (i) a candidate agent; (ii) a first biological sample comprising at least one first cell; and (iii) a recombinant nucleic acid construct comprising a nucleotide sequence that encodes an ABC transporter polypeptide and a promoter that is operably linked to a reporter gene, under conditions and for a time sufficient to detect transcription or expression of the reporter gene;
(b) comparing a level of reporter gene transcription or expression in the first cell with a level of reporter gene transcription or expression in a second cell in a control sample that has not been contacted with the candidate agent, wherein an increased level of reporter gene transcription or expression in the first cell in the presence of the candidate agent relative to the level of reporter gene transcription or expression in the second cell in the control sample that has not been contacted with the candidate agent indicates that the candidate agent is capable of modulating ABC transporter transcription or expression, thereby modulating catecholaminergic cell toxicity in the cell.

57. The method of claim 56 wherein the ABC transporter gene is an ABCG4 gene and wherein the recombinant nucleic acid construct comprises a nucleotide sequence having at least 80% identity with a sequence as set forth in SEQ ID NO:3.

58. The method of claim 56 wherein the ABC transporter gene is an ABCC5 gene and wherein the recombinant nucleic acid construct comprises a nucleotide sequence having at least 80% identity with a sequence as set forth in SEQ ID NO:1.

59. The method of claim 56 wherein the re porter gene is selected from the group consisting of chloramphenicol acetyltransferase, firefly luciferase, beta-galactosidase, and green fluorescent protein.

60. A method for identifying an agent that is capable of modulating catecholaminergic cell toxicity, comprising:
(a) contacting (i) a candidate agent and (ii) a first biological sample comprising at least one first cell, under conditions and for a time sufficient to detect catecholaminergic cell toxicity in the first cell, wherein the first cell is capable of expressing at least one ABC transporter polypeptide;
(b) comparing a level of catecholaminergic cell toxicity in the first cell with a level of catecholaminergic cell toxicity in at least one second cell in a control sample that has not been contacted with the candidate agent, wherein a reduced level of catecholaminergic cell toxicity in the first cell relative to the level of catecholaminergic cell toxicity in the second cell in the control sample that has not been contacted with the candidate agent indicates that the agent is capable of modulating catecholaminergic cell toxicity.

61. The method of claim 60 wherein the ABC transporter polypeptide is selected from the group of an ABCC5 transporter polypeptide and an ABCG4 transporter polypeptide.

62. The method of claim 60 wherein modulating catecholaminergic cell toxicity comprises modulating expression of the ABC transporter polypeptide.

63. The method of claim 62 wherein expression of the ABC transporter polypeptide is increased.

64. The method of claim 60 wherein modulating catecholaminergic cell toxicity comprises modulating a functional activity of the ABC transporter polypeptide.

65. The method of claim 64 wherein the functional activity of the ABC transporter polypeptide is increased.

66. The method of claim 64 wherein the functional activity of the ABC transporter polypeptide comprises modulating transport or translocation of a substrate across a membrane of the first cell.

67. The method of claim 64 wherein the functional activity of the ABC transporter polypeptide comprises modulating extrusion of a substrate from the first cell.

68. The method of either claim 66 or claim 67, wherein the substrate comprises a catecholamine, or a conjugate thereof.

69. The method of either claim 66 or claim 67, wherein the substrate comprises dopamine, or a conjugate thereof.

70. The method of claim 64 wherein the functional activity of the ABC transporter polypeptide comprises ATP hydrolysis.

71. The method of claim 62 wherein modulating expression of the ABC transporter comprises altering degradation of the ABC transporter polypeptide.
